(19) United States
(12) Patent Application Publication
(10) Pub. No.: US 2007/0157931 A1
(43) Pub. Date: Jul. 12, 2007

(54) System and method for optimized delivery of an aerosol to the respiratory tract

(76) Inventors: Richard Parker, Decatur, GA (US); Robby Sanders, McMinnville, TN (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/484,920

(22) Filed: Jul. 11, 2006

Related U.S. Application Data
(60) Provisional application No. 60/698,196, filed on Jul. 11, 2005.

(51) Int. Cl. A61M 16/00; A62B 7/00

(52) U.S. Cl. 128/204.23; 128/204.21

(57) ABSTRACT

The present disclosure relates to systems, methods, and devices for controlling delivery of aerosolized formulations to patients in need of treatment, which optimizes aerosol deposition to the respiratory tract of the patient and can be adapted for use in spontaneously breathing patients or in those requiring mechanical ventilation.

### CROSS-REFERENCE TO RELATED APPLICATIONS

This application claims priority to copending U.S. provisional patent application Ser. No. 60/698,196, entitled “System and Method for Improved Delivery of an Aerosol to the Respiratory Tract” filed on Jul. 11, 2005; which is entirely incorporated herein by reference.

### STATEMENT REGARDING FEDERALLY SPONSORED RESEARCH OR DEVELOPMENT

This invention was made with government support under Contract Numbers 1R41HL068393-01A1 and R44HL065791 both awarded by the National Heart, Lung and Blood Institute. The government has certain rights in the invention.

### BACKGROUND

Aerosol Delivery to Mechanically Ventilated Patients

Aerosol delivery of medications to patients on mechanical ventilators as currently practiced is inefficient. Aerosol delivery of medications to such patients may be affected by the nature of the lung disease, ventilator type, ventilator settings, gas composition and pressure, humidity in the ventilator circuit, drug formulation, the type and location of aerosol delivery device, airflow properties, and the time during the breath cycle that nebulization is initiated and stopped. Several attempts have been made to maximize aerosol delivery to the lungs and minimize waste without disrupting normal airway architecture.

Differences between continuous and breath-actuated aerosol delivery are especially pronounced in vivo, where breath actuation has been shown to increase delivery by 4-fold. Most aerosol medications are delivered to patients on mechanical ventilators via metered dose inhalers (MDIs). This approach has the advantages of insensitivity to ventilator settings and the ability to limit delivery to the inspiratory portion of the respiratory cycle, but it requires manual delivery (usually by a respiratory therapist) which introduces inefficiencies and variability inherent in human performance.

A recent survey summarized how albuterol is delivered in neonatal intensive care units across the United States. The study pointed out the differences in how medications are delivered among hospitals and confirm that wet aerosol medications are delivered predominantly by MDIs but also frequently by nebulizers. Delivery of fragile biological formulations, such as protein, DNA, or pulmonary surfactant formulations to the lungs is generally accomplished using wet solutions. Thus, these solutions will probably have to be delivered by nebulizer.

Devices that combine nebulizer technologies with strategies that allow aerosol generation during inspiration only have not, to date, proven readily adaptable for use in mechanically ventilated patients. Available aerosol devices do not allow automatic selection of aerosol delivery at any desired portion of inspiration.

While some nebulizer technologies such as jet nebulizers and even low-flow jet nebulizers may be suitable for aerosol delivery to ambulatory patients, many of them are not readily integrated into a ventilator circuit and may not be suitable for delivery of sensitive or fragile biological formulations. The additional airflow that would be introduced into the ventilator circuit would require caregiver intervention to ensure appropriate ventilation. Ultrasonic nebulizers can generate aerosols of select gene-based formulations without degrading the DNA, but these devices can generate substantial heat, so that the cycle time must be minimized. Many DNA formulations must be prepared at low concentrations, thus, requiring large volumes and long cycle times to deliver therapeutic doses. The long cycle times may render standard jet nebulizers inappropriate for aerosol gene therapy due to the nature of their operation and continuous recycling of the liquid formulation.

Vibrating mesh nebulizers generate aerosols by moving a liquid formulation through a diaphragm with precision placed micron-sized pores either by vibrating the diaphragm or vibrating a ring around the diaphragm at sub-ultrasonic frequencies. Essentially no additional air flow is introduced into the breathing circuit, and the aerosol generation process itself does not expose the liquid formulation in the reservoir to the nebulization process until the product is aerosolized out of the reservoir for inhalation. Thus, such nebulizers may be appropriate for use with fragile biological formulations, such as DNA-based or pulmonary surfactant formulations.

The most commonly used devices for delivering drugs to the lungs by aerosol are inefficient and may leak substantial amounts of drug into the environment. Efficiency and containment are not necessary priorities when the medicines delivered are cheap and environmental risk is negligible (e.g. albuterol). However, as next generation aerosolized medications including DNA and protein-based therapeutics, come into clinical use, development of efficient delivery technologies will be driven by considerations of cost, convenience for the patient and an environmental imperative.

Gene- and protein-based products for alpha-1 antitrypsin (AAT) deficiency and chronic obstructive pulmonary disease (COPD) and gene-based products for pulmonary hypertension are both being developed for aerosol delivery. Other gene-based therapies are also being developed for the treatment of patients who are critically ill with pulmonary disease, such as acute respiratory distress syndrome (ARDS).

Surfactants are substances naturally produced in the lungs essential for proper breathing, alveolar stability and gas exchange. Dysfunction or lack of surfactants is associated with serious respiratory diseases. As surfactant development occurs during the later stages of gestation, surfactant deficiency is observed in prematurely born infants and is associated with infant respiratory distress syndrome (IRDS), a life-threatening and costly disorder. Surfactant dysfunction also occurs in adults secondary to a number of traumatic events, such as acute lung injury (ALI) and ARDS. Surfactants have also demonstrated a statistical benefit in treating bronchopulmonary dysplasia (BPD), a syndrome associated with the prolonged use of mechanical ventilation and oxygen supplementation affecting about 10,000 to 25,000 babies per year in the United States alone, with the treatment of each patient costing up to $250,000. Meconium aspiration syndrome (MAS), in which the meconium inactivates lung surfactants and produces chemical irritations and infections of lung tissue, affects 30,000 infants worldwide per year.

Existing surfactant delivery methods are limited to direct injection of a surfactant solution through the trachea into the patient's lungs, called the “wet” or instillation method. This method carries critical shortcomings including, but not limited to the following: the introduction of a relatively large volume of liquid into already compromised lungs, which can block the air circulation and further compromise the already hypoxic patient and is particularly critical in neonatology; the length of the procedure, which takes an average of about 45 minutes, and the waste, via exhalation, of a considerable portion of the relatively expensive surfactant formulation. Thus, an efficient method of delivering surfactant formulations to patients that does not obstruct the airway spaces may provide a safer and more effective therapy with improved ease of administration and provide considerable cost savings.

### SUMMARY

Briefly described, the present disclosure provides systems, devices, and methods for optimizing delivery of an aerosolized formulation to the respiratory tract of a patient in need of treatment. In an embodiment, a system according to the present disclosure includes the following: an inspiration sensor for detecting initiation and cessation of inspiration and detecting the rate and amount of a gas flowing past the sensor, an aerosol generator (e.g., a nebulizer or metered dose inhaler (MDI)) for aerosolizing and releasing a formulation to the respiratory tract of the patient; and a computer system for implementing an aerosol controller system and in communication with (e.g., coupled to) the inspiration sensor and the aerosol generator.

In embodiments of the system, the computer system/aerosol controller system performs the following functions: receiving information from the inspiration sensor; processing information received from the inspiration sensor to determine at least one respiration parameter, as described below; and automatically determining the desired time during a respiration cycle for initiation and cessation of aerosol release based upon one or more respiration parameters and on one or more delivery parameters. The delivery parameters, as described in greater detail below, include inputted delivery parameters and calculated delivery parameters. In exemplary embodiments, the controller system determines calculated delivery parameters based on one or more respiration parameters and optionally one or more inputted delivery parameters. The computer system communicates with the aerosol generator to activate and terminate the release of aerosolized formulation based on the calculated delivery parameters and/or respiration parameters; and continually repeats the process while automatically adjusting for any changes in the respiration parameters or delivery parameters until a desired amount of formulation has been delivered to the patient.

In embodiments of the present disclosure, the system also includes a waste sensor in communication with the computer system for detecting an amount of waste. In such embodiments the computer system receives information from the waste sensor, and the aerosol controller system processes information received from the waste sensor, determines a waste percentage, and automatically re-calculates delivery parameters and adjusts the timing of initiation and cessation of aerosol release if the waste percentage exceeds a pre-determined waste tolerance threshold.

In embodiments of the present disclosure, the methods of the present disclosure are performed by an aerosol controller logic, described below and also sometimes referred to herein as an aerosol controller system, which is configured to implement the methods of optimizing aerosol delivery according to the present disclosure. In embodiments, the aerosol controller logic is implemented in hardware, or software, or a combination of hardware and software. In some embodiments, the aerosol controller logic is stored on a computer readable medium. In embodiments the aerosol controller logic is implemented in a computer system, as described below.

Embodiments of methods for optimizing aerosol delivery to a patient according to the present disclosure, briefly described, include the following steps: detecting respiration data and determining respiration parameters from respiration data; acquiring inputted delivery parameters; determining calculated delivery parameters from respiration data and one or more inputted delivery parameters; determining the desired time during a respiration cycle for initiation and cessation of aerosol release based upon one or more respiration parameters and one or more calculated delivery parameters; communicating with an aerosol generator to activate and terminate the release of aerosolized formulation, and repeating the process, while continuing to collect data and automatically adjusting delivery parameters based on changing data, until the desired amount of formulation has been delivered. Embodiments of the methods of the present disclosure also optionally include detecting waste, calculating a waste percentage, and adjusting calculated delivery parameters based on detected waste.

Other aspects, methods, devices, features, and advantages of the present disclosure will be or become apparent to one with skill in the art upon examination of the following drawings and detailed description. It is intended that all such additional compositions, methods, features, and advantages be included within this description, be within the scope of the present disclosure, and be protected by the accompanying claims.

### BRIEF DESCRIPTION OF THE FIGURES

FIG. 1 is a block diagram of an embodiment of an exemplary aerosol controller system capable of performing the methods of the present disclosure implemented in software executed by the computer hardware architecture shown.

FIG. 2 is a schematic illustration of an embodiment of a device/system of the present disclosure for optimizing aerosol delivery to the respiratory tract in which the methods of the present disclosure for optimizing delivery of aerosol formulations may be implemented.

FIG. 3 is a flow diagram that illustrates an embodiment of an optimized aerosol delivery method according to the present disclosure that can be performed by the aerosol controller system shown in FIG. 1 and executed by the system shown in FIG. 2.

FIG. 4 is a flow diagram that illustrates an embodiment of an optimized aerosol delivery method according to the present disclosure.

FIG. 5 is a flow diagram that illustrates an embodiment of a method according to the present disclosure of determining average tidal volume for determining calculated delivery parameters for optimizing aerosol delivery to a patient.

FIG. 6 is a flow diagram that illustrates an embodiment of a method of monitoring waste according to the present disclosure and using waste monitoring to optimize delivery parameters for increasing efficiency of aerosol delivery to a patient.

FIG. 7 is a diagram of an embodiment of a hand-held aerosol delivery device of the present disclosure.

FIG. 8A is a cross sectional view of an embodiment of a hand-held aerosol delivery device of the present disclosure illustrating the inspiration airflow pathway. FIG. 8B illustrates the expiratory airflow pathway in the same device.

FIG. 9 is a schematic diagram of a test embodiment of the system of the present disclosure and a “test lung” used in Example 1.

FIGS. 10A and 10B are bar graphs illustrating a pilot assessment of the effects of aerosol delivery efficiency when starting aerosol production at different points relative to the beginning of inspiration and continuing for duty cycles (e.g., percent of time allotted for inspiration) of 25%, 37.5%, 50%, and 100% corresponding to 0.75, 1.125, 1.5, and 3 seconds of nebulization, respectively. The panels correspond to the fraction of nebulizer charge deposited in a test lung filter (FIG. 10A) and a waste filter (FIG. 10B). The dashed lines represent results during continuous nebulization.

FIG. 11 is a bar graph illustrating the effects of chase volume on aerosol delivery efficiency assessed through measures of isotope collected in the test lung and waste filters of the test system illustrated in FIG. 9 and calculations of rainout. Aerosol production was started at different points relative to inspiration and continued for 1.125 seconds (37.5% duty cycle) such that aerosol production was stopped at a point during the breath cycle that the indicated volumes of air continued into the lungs. (The “*” indicates values that are statistically different from corresponding values obtained with a 331 ml chase volume).

FIG. 12 is a graph illustrating the effects on aerosol delivery efficiency using a jet nebulizer, showing measures of isotope collected in a test lung and waste filter.

FIG. 13 is a bar graph illustrating the effects of nebulizer control on delivery efficiency of different sized aerosol particles using vibrating mesh nebulizers. Aerosol production was started in synch with inspiration and continued for 1.125 seconds (37.5% duty cycle) using different vibrating mesh-type nebulizers with pores machined to generate aerosols of the indicated sizes. Shown are the fraction of nebulizer charge collected in test lung filters, waste filters, and rainout collected from the nebulizer outlet and ventilator tubing, and the sum total of these normalized counts. (The symbols used have the following meanings: “*” indicates statistically different from all other values in series; “+” indicates statistically different from all other test lung filter values except those designated with this sign; “ˆ” indicates statistically different from all other rainout values except those designated with this sign; “°” indicates statistically different from all other rainout values except those at 6.7 and 7.6 μm; “‡” indicates statistically different from all other rainout values except those at 9.3 μm and either 6.7 or 7.6 μm; and “†” indicates statistically different from waste at 11.7 μm).

FIG. 14A-D are bar graphs illustrating the effects of inspiratory:expiratory (I:E) ratios on delivery efficiency during continuous and phasic nebulization assessed through measures of test lung filter (crosshatched bars) and waste filter (solid bars) with deposition of 4.7 μm and 9.3 μm VMD aerosols. FIG. 14A) 4.7 μm aerosols at 1:1 I:E; FIG. 14B) 4.7 μm aerosols at 1:3 I:E; FIG. 14C) 9.3 μm aerosols at 1:1 I:E; FIG. 14D) 9.3 μm aerosols at 1:3 I:E.

FIG. 15 illustrates a schematic diagram of a test system used on sheep for the experiments described in Examples 2-4.

FIG. 16 is a bar graph illustrating the comparison of normal and elevated airway pressure on the efficiency of delivery of an aerosol formulation to ventilated sheep.

FIG. 17 is a bar graph illustrating the comparison of normal and elevated airway pressure on the amount of waste of an aerosol formulation delivered to ventilated sheep.

FIG. 18 is a bar graph illustrating the ratio of lung counts to waste counts of the data from FIGS. 16 and 17.

FIG. 19 is a composite radioimage of ^{99m}Tc-DTPA and ¹³¹I-albumin deposition in the lungs of ventilated sheep during aerosol deposition as described in Example 4.

FIG. 20 illustrates radioisotope distributions in lung tissue of ventilated sheep after aerosol deposition as assessed from gamma energy emissions counting of lung tissue samples.

FIG. 21 compares the effects of aerosolization of a DNA-based lipoplex formulation by a jet nebulizer and vibrating mesh nebulizer on DNA integrity as described in Example 5.

FIG. 22 compares the effect of aerosol delivery of a DNA-based formulation via jet or vibrating mesh nebulizer on gene expression as described in Example 5.

### DETAILED DESCRIPTION

Definitions:

As used herein the term “respiratory drug” generally refers to any pharmaceutically effective compound used in the treatment of any respiratory disease and in particular the treatment of diseases such as asthma, bronchitis, emphysema, lung infection, cystic fibrosis, AAT deficiency, COPD, ARDS, IRDS, BPD, and MAS, among others. Useful “respiratory drugs” include, but are not limited to, those that are listed within the Physician's Desk Reference (most recent edition). Such drugs include, but are not limited to, beta adrenergic agonists which include bronchodilators including albuterol, isoproterenol sulfate, metaproterenol sulfate, terbutaline sulfate, pirbuterol acetate, salmeterol xinotoate, formotorol; steroids including corticosteroids such as beclomethasone dipropionate, flunisolide, fluticasone, budesonide and triamcinolone acetonide; peptide non-adrenergic non-cholinergic neurotransmitters and anticholinergics. Anti-inflammatory drugs used in connection with the treatment of respiratory diseases include steroids such as beclomethasone dipropionate, triamcinolone acetonide, flunisolide and fluticasone. Other anti-inflammatory drugs and antiasthmatics used include cromoglycates such as cromolyn sodium. Other respiratory drugs which would qualify as bronchodilators include anticholinergics including ipratropium bromide. Other useful respiratory drugs include leukotriene (LT) inhibitors, vasoactive intestinal peptide (VIP), tachykinin antagonists, bradykinin antagonists, endothelin antagonists, heparin furosemide, anti-adhesion molecules, cytokine modulators, biologically active endonucleases, recombinant human (rh) DNase compounds, alpha-1 antitrypsin, and disodium cromoglycate (DSCG).

The present disclosure is also intended to encompass the free acids, free bases, salts, amines and various hydrate forms including semi-hydrate forms of such respiratory drugs and pharmaceutically acceptable formulations of such drugs which are formulated in combination with pharmaceutically acceptable excipient materials generally known to those skilled in the art, preferably without other additives such as preservatives. In some embodiments, the drug formulations do not include additional components such as preservatives, which cause adverse effects. Thus, such formulations consist essentially of a pharmaceutically active drug and a pharmaceutically acceptable carrier (e.g., water and/or ethanol). However, if a drug is liquid without an excipient, the formulation may consist essentially of the drug, which has a sufficiently low viscosity that it can be aerosolized using a dispenser of the present disclosure. In other embodiments, drug formulations may include one or more active ingredients, a pharmaceutically acceptable carrier and/or excipient, as well as other compounds such as, but not limited to, emulsifiers, buffers, preservatives, and the like, as appropriate.

According to the present disclosure, respiratory drug also includes gene or protein therapy based formulations that include genetic material, as defined below. Respiratory drug also includes formulations including pulmonary surfactants, as defined below, including both natural and synthetic surfactants.

As used herein the term “formulation” generally refers to any mixture, solution, suspension or the like which contains an active ingredient and a carrier and has physical properties such that when the formulation is moved through a aerosol generator, as described herein, the formulation is aerosolized into particles which are delivered/inhaled into the lungs of a patient. The active ingredient may be any pharmaceutically active respiratory drug (as defined above), or diagnostic or imaging agent. The carrier may be any pharmaceutically acceptable flowable liquid that is compatible with the active agent. Useful drugs include respiratory drugs defined above, systemically-active drugs delivered to the airways, and useful diagnostics including those used in connection with ventilation imaging. The formulation may also comprise genetic material dispersed or dissolved in a carrier, where the genetic material (when in a cell of the patient) expresses a pharmaceutically active protein or peptide. Formulations are preferably solutions, e.g., aqueous solutions, ethanoic solutions, aqueous/ethanoic solutions, saline solutions, colloidal suspensions and microcrystalline suspensions. In embodiments, formulations can be solutions or suspensions of drug in a low boiling point propellant.

As used herein the term “waste” generally refers to that portion of formulation that is not deposited within the lung or, if desired, the larger airways of the lung/respiratory tract. Waste includes, but is not limited to, the following types of waste:

Rain-out: Waste in which generated aerosol droplets impact on the ventilator tubing, connectors, etc. (and, thus, do not enter the lungs and/or larger airways of the lung) prior to entering the lungs.

Wrap-around: Waste generated as aerosol droplets flow down the ventilator circuit exhale tubing during inspiration. This is generally caused by compression of the gas in the exhale tubing during the increase in gas pressure that inflates the lung or via airflow induced by a jet nebulizer during expiration.

Exhalation: This type of waste is exhaled aerosol droplets that entered the lung (or artificial airways) but did not deposit in the lung/airways.

Sputter volume: This type of waste is mostly associated with jet nebulizers. It is a sum of the nebulizer dead volume and the splatter volume created due to the action of the baffles on aerosol generation. This volume may evaporate over time and cause the fluid to become more concentrated.

As used herein the term “imaging composition” generally refers to a formulation to be delivered to the respiratory tract of a patient, which, after deposition in the patient, will allow for imaging of the patient's airways and other portions of the respiratory tract via an imaging device, including, but not limited to, MRI, X-ray, and CT.

As used herein the term “genetic material” generally refers to material which includes a biologically active component, including but not limited to nucleic acids (e.g., single or double stranded DNA or RNA or siRNA's), proteins, peptides, and the like.

As used herein the term “surfactant” or “pulmonary surfactant” generally refers to specific lipo-protein substances naturally produced in the lungs that are essential for proper breathing, alveolar stability and gas exchange. Pulmonary surfactants are surface-active agents naturally formed by type II alveolar cells that reduce the surface tension at the air-liquid interface of alveoli. Pulmonary surfactants are generally made up of about 90% lipids (about half of which is the phospolipid dipalmitoylphosphatidylcholine (DPPC)) and about 10% protein. At least four native surfactants have been identified. SP-A, B, C, and D. The hydrophobic surfactant proteins B (SP-B) and C (SP-C) are tightly bound to the phospholipids, and promote their adsorption into the air-liquid interface of the alveoli. These proteins are critical for formation of the surfactant film.

The term “surfactant” also includes currently available surfactant preparations, including, but not limited to, Survanta® (beractant), Infasurf® (calfactant), Exosurf neonatal® (colfosceril palmitate), Curosurf® (poractant alfa), Surfaxin® (lucinactant), Aerosurf® (aerosolized Surfaxin®), Vanticute® (lusupultide), Alveofact®) (bovactant), as well as preparations being developed.

As used herein, the term “host” includes humans and other living species that are in need of treatment and capable of being ventilated or of using a portable inhaler. In particular, the term “host” includes humans and mammals (e.g., cats, dogs, horses, chicken, pigs, hogs, cows, and other cattle).

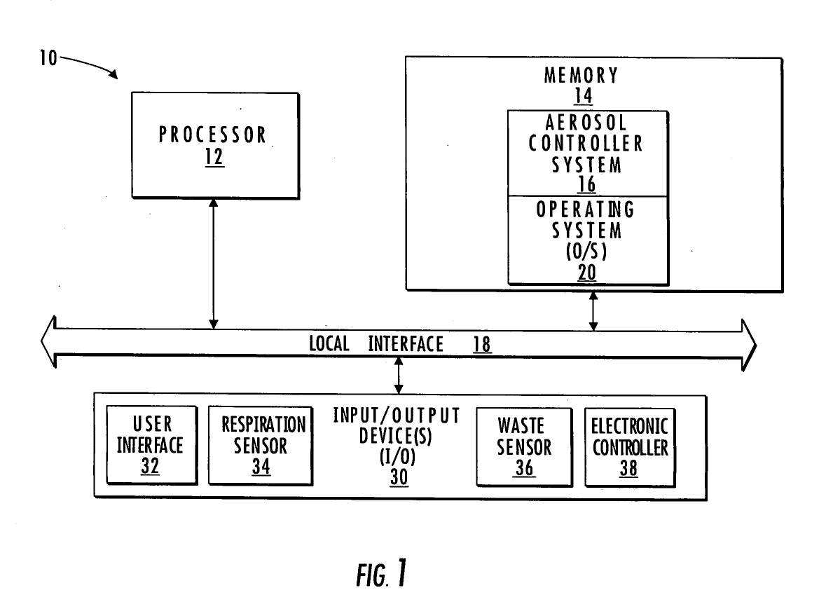

As used herein, the term “aerosol controller system,” also referred to herein as “aerosol controller logic,” indicates any system or program that can be implemented in software (e.g., firmware), hardware, or a combination thereof to perform the desired functions as set forth in this disclosure. In one example, the aerosol controller system is implemented in software, as an executable program, and is executed by a special or general purpose digital computer, such as a personal computer (PC; IBM-compatible, Apple-compatible, or otherwise), workstation, minicomputer, or mainframe computer which may be adapted to interface with other parts of the device of the present disclosure, including but not limited to an inspiration sensor, an exhalation sensor, and a nebulizer. An example of a general purpose computer that can implement the aerosol controller system of the present disclosure is shown in FIG. 1. In FIG. 1, the aerosol controller system is denoted by reference numeral 16.

Generally, in terms of hardware architecture, as shown in FIG. 1, the computer system 10 includes a processor 12, memory 14, and one or more input and/or output (I/O) devices 30 (or peripherals) that are communicatively coupled via a local interface 18. The local interface 18 can be, for example but not limited to, one or more buses or other wired or wireless connections, as is known in the art. The local interface 18 may have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 12 is a hardware device for executing software, particularly that stored in memory 14. The processor 12 can be any custom made or commercially available processor, a central processing unit (CPU), an auxiliary processor among several processors associated with the computer 10, a semiconductor based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80×86 or Pentium series microprocessor from Intel Corporation, a PowerPC microprocessor from IBM, a Sparc microprocessor from Sun Microsystems, Inc, or a 68xxx series microprocessor from Motorola Corporation.

The memory 14 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 14 may incorporate electronic, magnetic, optical, and/or other types of storage media. Note that the memory 14 can have a distributed architecture, where various components are situated remote from one another, but can be accessed by the processor 12.

The software in memory 14 may include one or more separate programs, each of which comprises an ordered listing of executable instructions for implementing logical functions. In the example of FIG. 1, the software in the memory 14 includes the aerosol controller system 16 in accordance with the present disclosure and a suitable operating system (O/S) 20. A nonexhaustive list of examples of suitable commercially available operating systems 20 is as follows: (a) a Windows operating system available from Microsoft Corporation; (b) a Netware operating system available from Novell, Inc.; (c) a Macintosh operating system available from Apple Computer, Inc.; (e) a UNIX operating system, which is available for purchase from many vendors, such as the Hewlett-Packard Company, Sun Microsystems, Inc., and AT&T Corporation; (d) a LINUX operating system, which is freeware that is readily available on the Internet; (e) a run time Vxworks operating system from WindRiver Systems, Inc.; or (f) an appliance-based operating system, such as that implemented in handheld computers or personal data assistants (PDAs) (e.g., PalmOS available from Palm Computing, Inc., and Windows CE available from Microsoft Corporation). The operating system 20 essentially controls the execution of other computer programs, such as the aerosol controller system 16, and provides scheduling, input-output control, file and data management, memory management, and communication control and related services.

The aerosol controller system 16 is a source program, executable program (object code), script, or any other entity comprising a set of instructions to be performed. When a source program, then the program needs to be translated via a compiler, assembler, interpreter, or the like, which may or may not be included within the memory 14, so as to operate properly in connection with the O/S 20. Furthermore, the aerosol controller system 16 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, and Ada.

The I/O devices 30 may include input devices, for example but not limited to, a keyboard, mouse, scanner, microphone, etc. Furthermore, the I/O devices 30 may also include output devices, for example but not limited to, a printer, display, etc. Finally, the I/O devices 30 may further include devices that communicate both inputs and outputs, for instance but not limited to, a modulator/demodulator (modem; for accessing another device, system, or network), a radio frequency (RF) or other transceiver, a telephonic interface, a bridge, a router, sensors, etc. In particular the I/O devices 30 may include a user interface 32 (which may include I/O devices such as a keyboard, mouse, display, etc.), a respiration sensor 34, a waste sensor 36, and an electronic controller 38 to control actuation and termination of aerosol formation by a nebulizer or MDI.

If the computer 10 is a PC, workstation, or the like, the software in the memory 14 may further include a basic input output system (BIOS) (omitted for simplicity). The BIOS is a set of essential software routines that initialize and test hardware at startup, start the O/S 20, and support the transfer of data among the hardware devices. The BIOS is stored in ROM so that the BIOS can be executed when the computer 10 is activated.

When the computer 10 is in operation, the processor 12 is configured to execute software stored within the memory 14, to communicate data to and from the memory 14, and to generally control operations of the computer 10 pursuant to the software. The aerosol controller system 16 and the O/S 20, in whole or in part, but typically the latter, are read by the processor 12, perhaps buffered within the processor 12, and then executed.

When the aerosol controller system 16 is implemented in software, as is shown in FIG. 1, it should be noted that the aerosol controller system 16 can be stored on any computer readable medium for use by or in connection with any computer related system or method. In the context of this document, a computer readable medium is an electronic, magnetic, optical, or other physical device or system that can contain or store a computer program for use by or in connection with a computer related system or method. The aerosol controller system 16 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

In an alternative embodiment, where the aerosol controller system 16 is implemented in hardware, the aerosol controller system can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The aerosol controller logic can be implemented, in one embodiment, as a single module or, in some embodiments, as a distributed network of modules, where one or more of the modules can be accessed by one or more applications or programs or components thereof.

In an embodiment, the electronic controller system and any necessary components of the computer system described above may be implemented in hardware and miniaturized with any necessary solid state electronics for integration into a portable device, such as a hand-held aerosol delivery device described in greater detail below.

General Description:

What is needed is a system for controlling delivery of aerosolized formulations to patients which substantially improves aerosol deposition, minimizes waste and environmental contamination and provides a substantially improved method for delivering gene- and protein-based formulations and other biological formulations, such as pulmonary surfactants. What is also needed is a device as described above that can be adapted for use in spontaneously breathing patients or in those requiring mechanical ventilation, ensures minimal contamination of the environment, and can aerosolize biological formulations without diminishing the therapeutic activity.

What is also needed is a device to allow timed delivery during select intervals of inspiration, where fragile biological formulations can be effectively delivered intact by aerosol to lung airspaces in a highly efficient, targeted manner, resulting in an improved therapeutic ratio with minimal waste and without environmental contamination.

The system of the present disclosure includes a controller mechanism to control aerosol delivery of medications to the respiratory tract of patients. The system of the present disclosure is not a nebulizer per se, but rather is an electronic controller that controls the timing of aerosol generation such that aerosols are only generated during select intervals of the respiratory cycle. By so doing, aerosol delivery to the lungs can be greatly enhanced and waste can be minimized, as desired. The device and methods of the present disclosure is also unique in that it will be able to automatically control and optimize drug delivery to both mechanically-ventilated and ambulatory, spontaneously breathing patients.

Depending on the therapeutic regimen of the patient, the system controls either nebulizers or MDIs in a manner that can provide maximal drug delivery to the lung, relative to a desired efficiency setting. Some embodiments of the present disclosure are integrated into a device including nebulizers and/or MDI's with an integrated electronic aerosol controller system according to the present disclosure.

In one embodiment, the device of the present disclosure is adapted for delivery of aerosol formulations to ventilated patients using nebulizers including, but not limited to, jet, vibrating mesh, and ultrasonic nebulizers. In another embodiment, the device is adapted for MDI use in ventilated patients by controlling MDI actuation. In another embodiment of the disclosure, the device is adapted for delivery of aerosol formulations to spontaneously breathing patients. In certain aspects, the device for delivery to spontaneously breathing patients is a handheld, portable device. In embodiments of the present disclosure, the device automatically controls the delivery of aerosolized formulations to the respiratory tract of patients, and automatically adjusts various parameters of the device in response to changing conditions.

In aspects of the disclosure, the device enables selection of points in the respiration cycle to start and stop aerosol formation to maximize deposition of an aerosolized formulation to the respiratory tract of a patient. The device of the present disclosure increases the efficiency of aerosol deposition in the lungs for imaging compositions, respiratory drugs, pulmonary surfactants, proteins and/or gene-based medications. It has the potential ability to allow greater than about 80%, greater than about 85%, greater than about 90%, or greater than about 95% of the aerosol to be deposited in the lungs and/or allow a maximal deposition of an aerosol over a given time period.

To do this, the system of the present disclosure calculates tidal volume (e.g., from the air flow rate and/or volume and time) and initiates and terminates nebulization at specific points during the respiratory cycle, based on certain calculated parameters, to allow the remaining portion of the tidal volume to act as a chase-volume for the aerosol. Embodiments of the system of the present disclosure allow multiple drugs to be administered in serial, alternating or simultaneous manner by utilizing multiple aerosol generators. In exemplary embodiments of a system of the present disclosure, inputted and calculated delivery parameters including medicine, dose, time, chase volume, etc. can be logged and stored for later use and/or for reference.

In some embodiments of the disclosure, the device can be adjusted among various efficiency settings to tailor the efficiency to the particular circumstances. In aspects of the disclosure, various parameters of the device can be adjusted (either manually or automatically) to target aerosols to selected sections of the airway tree (e.g., at specific size airway branches). In yet other embodiments of the disclosure, the device monitors exhaled waste and can automatically adjust various parameters to reduce waste and increase efficiency. These and other embodiments of the present disclosure will be described in greater detail below.

In the following, various embodiments of systems and methods are described in detail. Although specific embodiments are presented, those embodiments are mere example implementations of the disclosed systems and methods and it is noted that other embodiments are possible. All such embodiments are intended to fall within the scope of this disclosure.

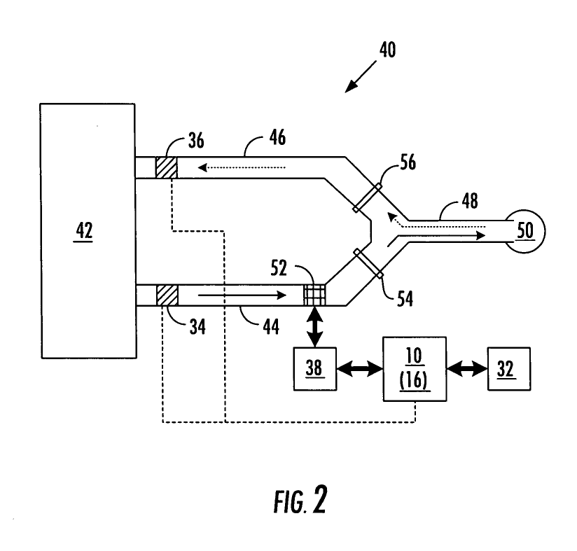

One embodiment of the device of the present disclosure is shown schematically in FIG. 2. Although the system illustrated in FIG. 2 is adapted for use with a ventilator 42, the various components of the device can also be included in a device adapted for use with spontaneously breathing patients. FIG. 2 illustrates an aerosol delivery system 40 that includes a ventilator 42, an inspiratory pathway/tubing 44, and expiratory pathway or tubing 46, and an intubation tube 48 which is inserted into the airway of a patient 50. The solid arrow represents inspiratory airflow (e.g., during inhalation), while the broken arrow represents expiratory airflow (e.g., during exhalation). The system 40 also includes one way airflow valves 54 and 56 to prevent exhaled air from flowing down the inspiratory pathway and vice versa. In some embodiments one way airflow valves will also or alternatively be incorporated into the ventilator 42. In embodiments, the system may include additional one way airflow valves in the ventilator 42 and/or the airflow pathways/tubing. It will be understood by one of skill in the art that airflow valves 54 and 56, and other components of the system 40 described below, may be located in various areas of the system 40, and are not limited to the locations shown in the embodiment illustrated in FIG. 2.

The system 40 further includes an inspiration sensor 34 to detect the initiation and cessation of inspiration/inhalation. In certain embodiments, the inspiration sensor 34 is a pressure sensor. In other embodiments, the inspiration sensor is a flow sensor 34, and in yet other embodiments the inspiration sensor 34 may include both pressure and flow sensors. In yet other embodiments, multiple inspiration sensors 34 can be implemented to detect various gasses. In certain embodiments of the present disclosure, the information from the inspiration sensor is communicated to an aerosol controller system 16 (which may be implemented in a computer system 10, as shown in FIG. 1), which processes the information from the inspiration sensor 34 and calculates various respiration parameters. “Respiration parameters”, as used herein includes, but is not limited to, parameters such as length of inspiration, peak inspiratory pressure, tidal volume, and length of the respiration cycle. In embodiments of the disclosure, the aerosol controller system records and logs information received and processed from the inspiration sensor and calculates average respiration parameters. The aerosol controller system 16 then uses the calculated averages to calculate other parameters and to predict parameters of the next breath.

The computer system 10 including electronic controller system 16 is also in communication with a user interface 32 where a user (e.g. a doctor, nurse, clinician, respiratory therapist, etc.) can input various parameters, including, but not limited to, patient-specific parameters (e.g., identification information, height, weight, illness, etc.), airway/equipment-specific parameters (e.g., endotracheal or intubation tube diameter, length, and placement from bronchi), formulation-specific parameters (e.g. drug specifications, volume/dosage to be delivered, particle size of the aerosolized formulation, order of delivery for multiple drugs, etc.), and other delivery-specific parameters such as the desired location in the respiratory tract for delivery, a desired delivery efficiency setting, an estimated chase volume, an estimated gas velocity, and the length of time to complete delivery of a desired volume/dose of formulation. Collectively, these parameters are sometimes referred to herein as “inputted delivery parameters.” The aerosol controller system 16 can then use the inputted delivery parameters and the respiration parameters to calculate optimized delivery parameters, as discussed in greater detail below.

As shown in FIG. 2, the system 40 also includes an electronic controller 38 that allows the aerosol controller system 16 (implemented in computer system 10) to control the actuation and termination of aerosol generation by an aerosol generator 52 housed in the inspiratory pathway 44. The aerosol generator 52 may be an MDI or one of various types of nebulizers discussed above. In an exemplary embodiment, aerosol generator 52 is a vibrating mesh nebulizer.

In some embodiments, the aerosol delivery system 40 also includes a waste sensor 36 housed in the expiratory pathway 46 for detecting and monitoring an amount of waste, as defined above. In preferred embodiments of the present disclosure, the waste sensor 36 detects exhaled waste, or a combination of exhaled and wrap-around waste, as defined above. In embodiments of the disclosure, the aerosol controller system 16 (implemented in computer system 10 in FIG. 2) receives information from the waste sensor and determines if the amount of exhaled waste exceeds a waste tolerance threshold. The waste tolerance threshold will vary depending on the formulation to be delivered or the efficiency setting, as more or less waste may be acceptable depending on the formulation to be delivered or the selected efficiency setting. For instance, if the formulation is an inexpensive respiratory drug that has little to no environmental effects, the waste tolerance threshold would likely be higher than for an expensive, fragile respiratory drug containing genetic material or surfactants. In situations where waste monitoring is critical, the waste tolerance threshold is desirably between about 0% and about 20% of the aerosolized formulation. In embodiments of the device the aerosol controller system automatically adjusts calculated delivery parameters based on the information from the waste sensor.

In yet other embodiments of the disclosure, the aerosol controller system uses information from the inspiration sensor and the calculated respiration parameters, optionally in combination with other parameters, such as delivery parameters and waste conditions (as discussed in greater detail in the flow charts below), to determine the desired timing for the initiation and termination of release of an aerosolized formulation. The delivery parameters include, but are not limited to, inputted delivery parameters and calculated delivery parameters. Inputted delivery parameters, which include the various parameters discussed above, are inputted into the aerosol controller system by a user (e.g., a clinician) via a user interface 32. “Calculated delivery parameters”, on the other hand, are calculated by the aerosol controller system based on one or more respiration parameters and one or more inputted delivery parameters or waste parameters. The calculated delivery parameters include, but are not limited to, a calculated and/or optimized chase volume, a calculated and/or optimized gas velocity and an optimized particle size. It is also contemplated that the calculated delivery parameters may be calculated and/or estimated by a user and manually inputted into the aerosol controller system, in which case such delivery parameters would also be inputted delivery parameters. For instance, a clinician could use patient-specific parameters and a look-up table to calculate an airway volume based on a desired target airway and could add that volume to equipment connection volume parameters (e.g., intubation tube volume) to determine the portion (chase volume) of the tidal volume that must be delivered/inhaled after stopping aerosol production to improve the opportunity for all particles to reach their airway target. In other words, the estimated chase volume could be subtracted from an estimated tidal volume to allow determination of an estimated optimal time to stop aerosol production. Thus, the user can manually enter such parameters or the aerosol controller system can automatically calculate such parameters.

Also, the aerosol controller system can initially calculate/estimate such delivery parameters based on inputted delivery parameters such as patient-specific parameters and empirical look-up tables, and then later re-calculate such parameters based on continuing input from the respiration sensor and optional waste sensor to provide continuously optimized delivery parameters. The aerosol controller system uses the calculated delivery parameters, the inputted delivery parameters, and the respiration parameters each with or without waste monitoring, as discussed below, to determine the desired timing for the initiation and cessation of the release of an aerosolized formulation to be delivered to the respiratory tract of a patient.

In embodiments of the device of the present disclosure, the aerosol controller system communicates with an aerosol generator to initiate release of a formulation and to terminate release of the formulation. In certain embodiments of the device, the aerosol generator is a nebulizer, including but not limited to, jet nebulizers, low-flow jet nebulizers, and vibrating mesh nebulizers. In other embodiments of the device, the aerosol generator is a metered dose inhaler. In some embodiments of the present disclosure multiple aerosol generators can be inserted to allow multiple drugs to be administered in serial, alternating or simultaneous manner. In yet other embodiments of the disclosure, the aerosol generator can produce particles of different sizes in order to target deposition of the aerosolized formulation to a certain portion of the respiratory tract. Some of these embodiments will be discussed in greater detail below.

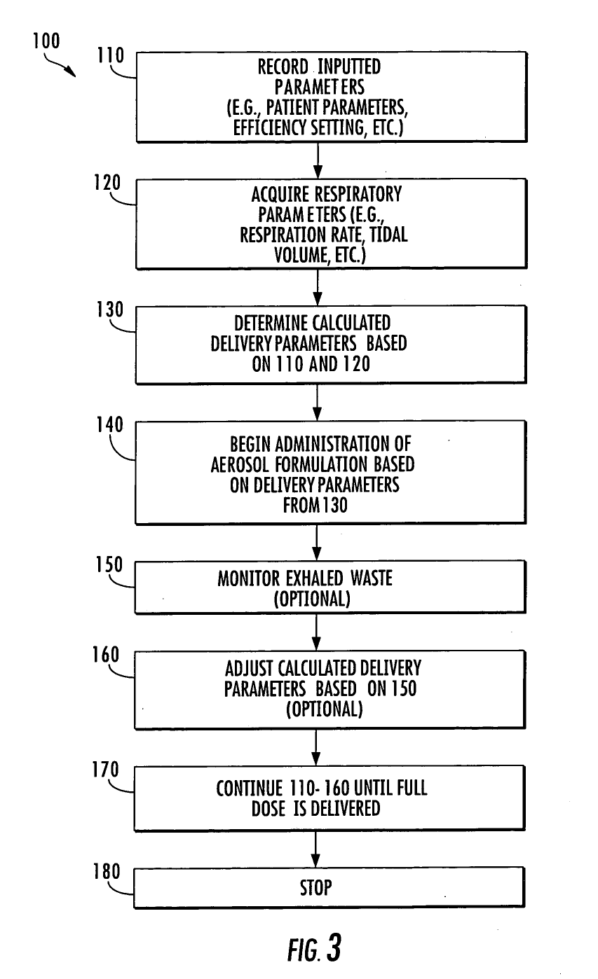

The flow charts shown in FIGS. 3-6 generally depict the embodiments of the device and method of delivering an aerosolized formula to the respiratory tract of a patient as described above. For the purposes of illustration only, and without limitation, embodiments of the present disclosure will be described with particular reference to the below-described methods. Note that not every step in the process is described with reference to the process described in the figures hereinafter. Therefore, the following optimization and delivery process is not intended to be an exhaustive list that includes every step useful or required for the delivery of an aerosol formulation to a patient. Additionally, it will be understood by one of skill in the art that the steps described below and illustrated in the flow charts of FIGS. 3-6 do not necessarily have to be performed in the order presented and can be performed in a different order where logically possible. Additionally, some steps may be performed continuously where logically possible.

FIG. 3 is a flow chart illustrating a general embodiment of a method 100 of optimizing aerosol delivery to the respiratory tract of a patient according to the present disclosure. In step 110 a user inputs delivery parameters, including, but not limited to, patient-specific parameters, formulation parameters, airway connection parameters, a desired efficiency setting, and the like as appropriate. In step 120 the system acquires respiration parameters (e.g., respiration rate, tidal volume, etc.) and uses the respiration parameters and inputted delivery parameters (step 130) to determine calculated delivery parameters (e.g., calculated chase volume, timing of delivery, etc.). The system then initiates and terminates aerosolization to administer a formulation to a patient based on the calculated delivery parameters. Note, however, that administration of the formulation can be started before acquiring respiration parameters, based on estimated delivery parameters (either manually inputted or automatically calculated by the aerosol controller system), and then optimized based on respiration parameters and re-calculated delivery parameters. Optionally, as illustrated in step 150, exhaled waste can be monitored and used to adjust calculated delivery parameters (step 160). Steps 110-160 are continued until the full dosage of formulation is delivered to the patient at which point nebulization and delivery control are stopped and all pertinent data and calculated values logged.

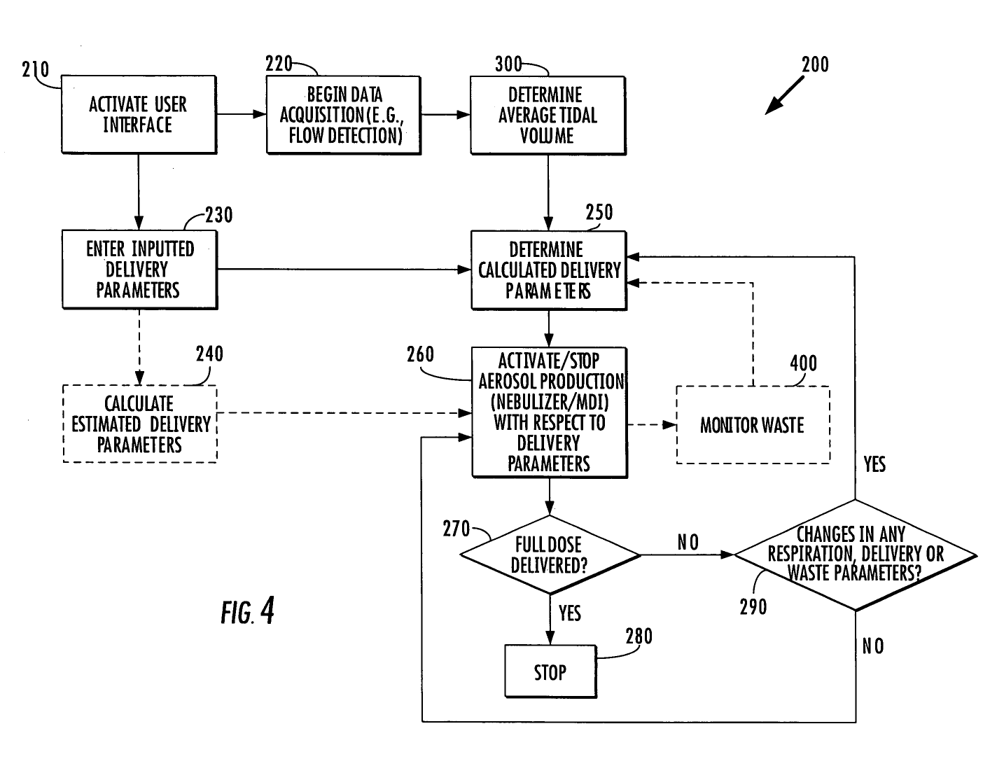

FIG. 4 is a more detailed illustration of an embodiment of a method 200 of optimizing delivery of an aerosol formulation according to the present disclosure. Optional or alternative steps/embodiments are shown by broken lines. In step 210 the user interface is activated, and then inputted delivery parameters are entered by a user (230). In a preferred embodiment, data is acquired, such as respiration parameters, as shown in step 220, and used to determine an average tidal volume (300). Additional details regarding embodiments for determining average tidal volume are presented in FIG. 5, discussed below. The average tidal volume from step 300 and inputted delivery parameters from step 230 are used to determine calculated delivery parameters (250). The calculated delivery parameters are then used in step 260 to determine the timing for activation/termination of aerosol production by the aerosol generator.

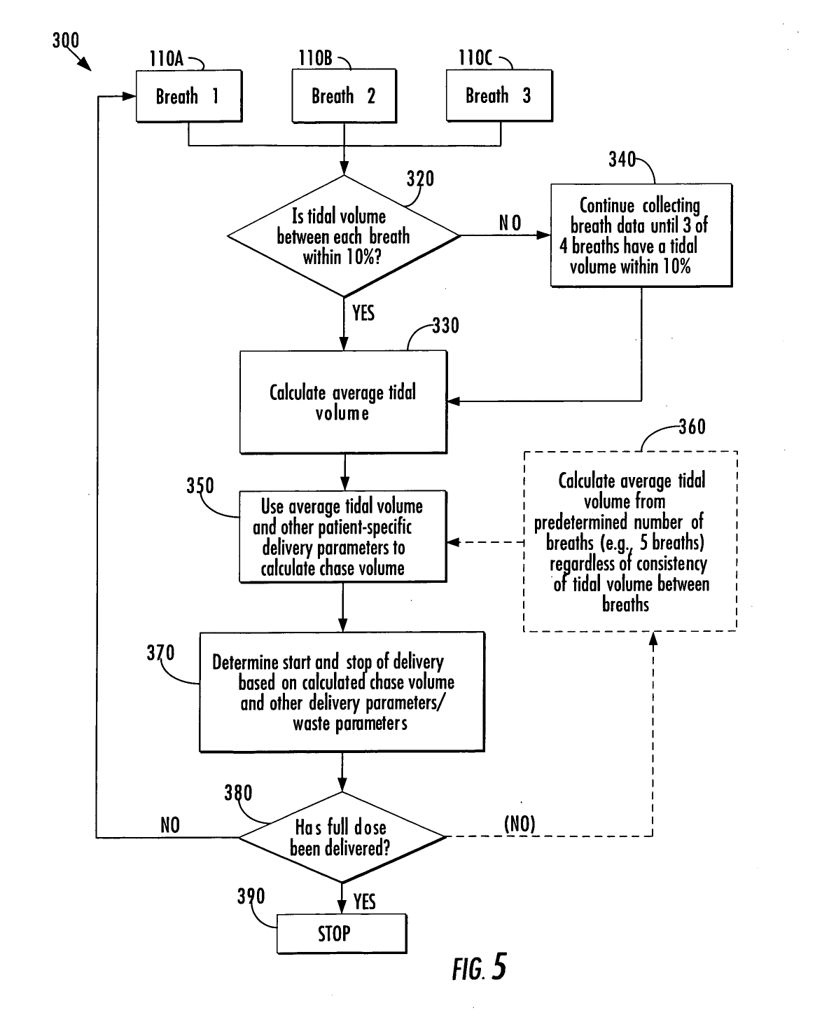

After aerosol delivery has begun, optionally, waste is monitored (400). Additional details regarding embodiments for determining average tidal volume are presented in FIG. 6, discussed below. Data acquired from waste monitoring (400) is also used in determining calculated delivery parameters. In an exemplary embodiment, calculated delivery parameters are continuously re-calculated based on any changes in respiration parameters, waste, or delivery parameters.

Optionally, before delivery of the aerosol formula is initiated, delivery parameters can be estimated based on the inputted delivery parameters and known look up tables and formulas, as shown in step 240. Step 240 can be performed manually by a user and inputted or can be automatically calculated by the aerosol controller system. The estimated delivery parameters can be used to begin delivery of aerosol formulation (260). In such an embodiment, after delivery has begun based on estimated delivery parameters from 240, data acquisition (220) and optional waste monitoring (400) also begin, and the delivery parameters are re-calculated based on the acquired data (250).

After delivery of the formulation has begun, if the full dose has been delivered (270), then the process is terminated (280). If the full dose has not been delivered (270), then it is determined if any changes in respiration parameters, delivery parameters or waste parameters have occurred (290). If no changes have occurred, then the same calculated delivery parameters are used to activate/terminate aerosol production (260) to continue delivery of the formulation. If/when changes do occur in any of the respiration, delivery, or waste parameters, then the calculated delivery parameters are re-calculated (250) based on the new parameters. Data acquisition and re-calculation of delivery parameters (as needed with respect to changes) continues until the full dosage is delivered.

FIG. 5 is a flow chart representing an illustrative method 300 for determining an average tidal volume and using the average tidal volume to optimize delivery of an aerosol formulation to a patient. The method 300 for determining an average tidal volume can be used in method 200 for optimized delivery of an aerosol formula, illustrated in FIG. 4. In method 300, step 310A-C represents data acquisition for a specified number of breaths. For purposes of illustration only, 3 breaths are used, but any number can be specified. If the tidal volume of each breath is within 10% of the other 2 breaths (320) then the tidal volume of these 3 breaths is used to calculate the average tidal volume (330) and that data is written to the system for calculating delivery parameters. If the tidal volume of all three breaths is not within about 10%, then data acquisition is continued until 3 of 4 breaths have a tidal volume within about 10% of each other (340) and can be used to calculate average tidal volume (330). It should be noted that percentages other than 10% can be used as the threshold percentage for consistency data. For instance, the breaths can be within about 5%, within about 8%, within about 15%, within about 20%, within about 25%, and so on. Preferably, the threshold percentage is about 1% to 20%.

In an alternative embodiment, shown in step 360 (indicated by broken lines), the average tidal volume may be calculated from a predetermined number of breaths (e.g, 5 breaths) without regard to whether such breaths are within a certain volume percentage of each other.

The average tidal volume from step 330 or 360 is then used, in combination with other delivery parameters (e.g., patient-specific delivery parameters) to determine a calculated chase volume (350). The calculated chase volume from 350 is combined with other respiration and delivery parameters, and, optionally, waste data, to determine the optimized timing for starting and stopping delivery of an aerosolized formulation (370). If the full dose has been delivered (380) then the process is terminated (390). However, if the full dosage has not been delivered (380), then data acquisition continues and chase volume and other delivery parameters are recalculated as necessary and the process is repeated until the full dosage is delivered. It should be noted that in an exemplary embodiment, data acquisition is continuous even during delivery of the aerosol and when no changes in parameters occur.

FIG. 6 is a flow chart illustrating a method 400 of monitoring waste for optimizing delivery of an aerosol formulation according to methods of the present disclosure. The method 400 for monitoring waste can be used in method 200 for optimized delivery of an aerosol formula, illustrated in FIG. 4. In the method of waste monitoring 400, a calculated chase volume is determined (410) based on one or more patient-specific parameters, respiration parameters, and delivery parameters, as described above. The aerosol generator (e.g., nebulizer or MDI) is activated relative to the initiation of inspiration (420) with respect to the delivery parameters, aerosol production is continued until a sufficient volume has been given to allow adequate chase volume (430) to enter the lungs, and then aerosol production is stopped (440). Waste is monitored during inhalation for wrap-around waste and during exhalation (450) for exhaled waste. If the total dosage has been given, then the process is terminated (490).

If the total dosage has not been given then the process continues. If there was no waste present in the previous exhalation or if waste did not exceed a selected waste threshold (470), then aerosol delivery is initiated again (420) and the process continues until either the full dosage is delivered or until exhaled waste exceeds the selected threshold. When the waste exceeds the threshold (470), then the aerosol generation stop volume is reduced by 1% (or other selected percentage increment) of tidal volume (480), and delivery is initiated based on the re-calculated delivery parameters (420). Waste monitoring (450) continues, and the stop volume is reduced by about 1% of stop volume after each actuation until the exhaled waste falls below the waste threshold. The waste threshold may be set at any percentage depending upon the desired delivery efficiency. For instance, for very efficient delivery, the waste threshold is desirably set at about 0% to 20% waste. However, if efficiency is not as critical, and shorter delivery time is more important, the waste threshold may be set much higher, for instance about 50% to 75%. Additional detail about optional efficiency settings is provided below.

Various embodiments of the methods, systems, and devices of the present disclosure are described in greater detail below, but these descriptions are not intended to be limiting, and those of skill in the art will understand that other methods of implementing the present disclosure exist and are within the scope of this disclosure.

Additional Description of Various Embodiments

Device Embodiments

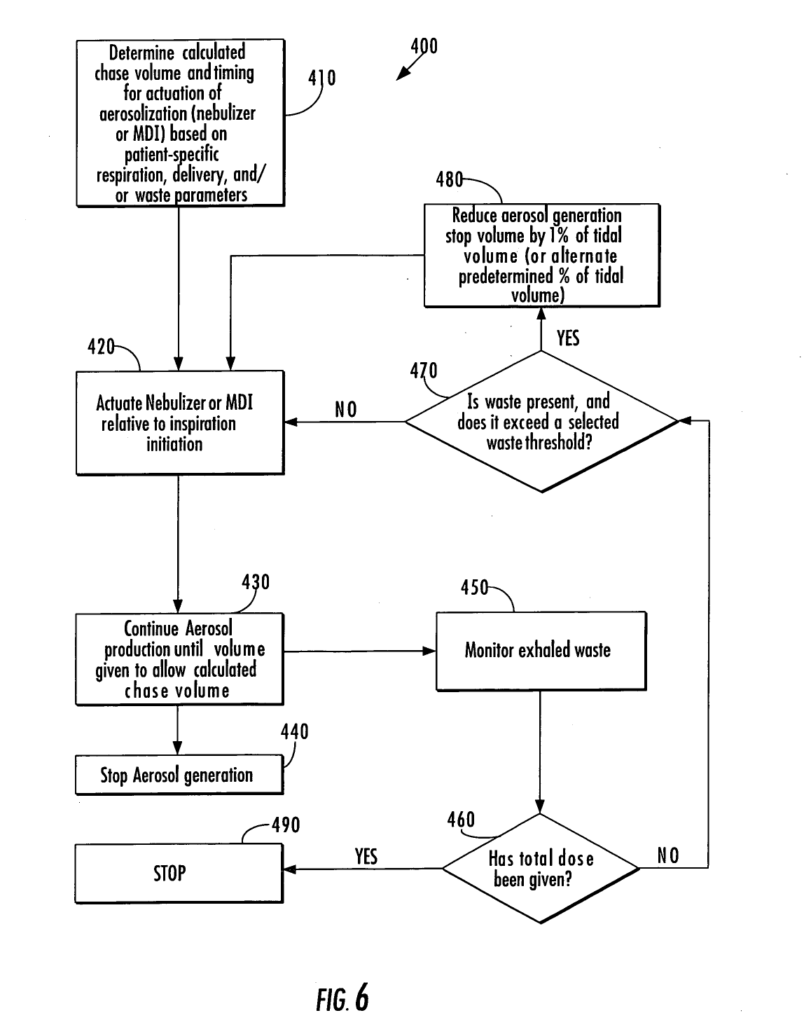

The methods illustrated in FIGS. 3-6 above can be implemented by an electronic controller system of the present disclosure, which can be integrated into a delivery system/device that may be in communication with or coupled to a ventilator system and utilizes a microcontroller or computer interface (such as shown in FIG. 2 and described above) or can be integrated with a hand-held MDI. An embodiment of a portable, hand-held device capable of performing the methods of the present disclosure is illustrated in FIG. 7.

The embodiment illustrated in FIG. 7 of a portable, hand-held device 500 includes an aerosol generator 510 for generating the aerosol formulation and a reservoir or compartment (not shown) for holding the formulation (or a slot for receiving a container containing the formulation to be delivered). The device also desirably includes a handle or grip 520, a power source 540 (e.g., a rechargeable battery pack).

The device also includes integrated electronics 530 (shown integrated into the handle piece). The integrated electronics 530 would include the aerosol controller system of the present disclosure and other hardware and/or software elements (e.g., a computer system as described above) necessary for implementing the aerosol controller system. The device also includes a mouthpiece 550, which, in embodiments, may be able to be rotated 180 degrees to alter a method of delivery (e.g., from optimized and breath-actuated to continuous). The airflow direction is indicated by arrow 570. The device also optionally includes a light indicator 560 for providing indications to a user, such as when to begin or end inhalation, when treatment is due, when treatment is complete, to resume an interrupted treatment, to increase or decrease inhalation rate/volume, and the like.

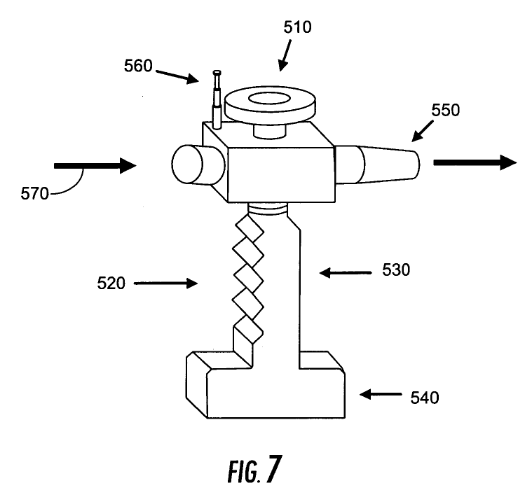

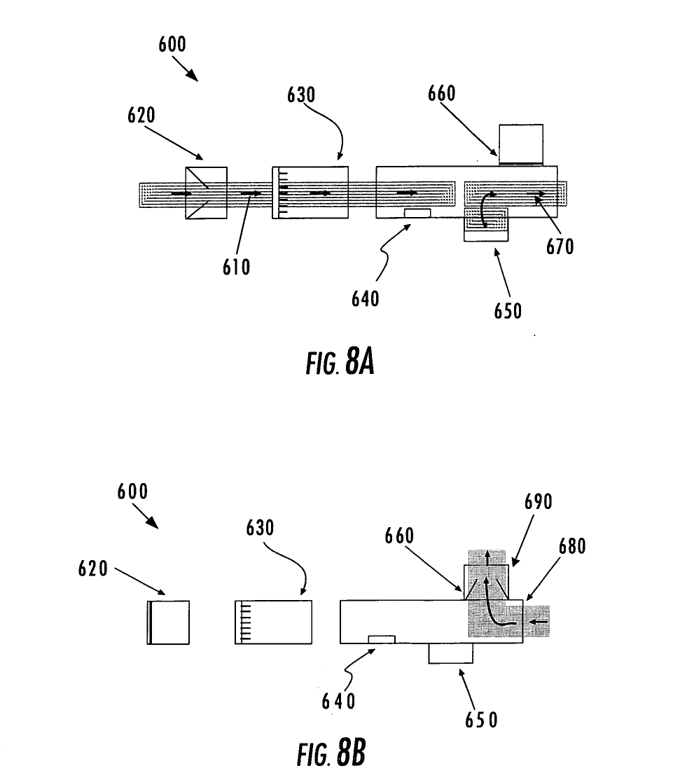

FIGS. 8A and B illustrate airflow through a cross-section of an embodiment of a portable device according to the present disclosure, such as the one shown in FIG. 7. FIG. 8A illustrates the airflow path, indicated by arrow 610, during inspiration. The air flows through a one-way inflow valve 620 and through an optionally adjustable air flow restrictor 630 (to assist a user in achieving the correct inspiratory rate/volume). Inhaled air then flows past an air flow sensor 640, which records respiration data for determination of respiration parameters. The air then passes the aerosol generator (e.g., nebulizer) 650 which introduces the aerosol bolus 670. The airflow then continues into the user's airways and lungs.

FIG. 8B illustrates the expiratory airflow pathway. The exhaled air 680 passes from a user's airways into the device, and out a one-way outflow valve 660 in an expiratory pathway 690. Optionally, the device also includes a waste sensor (not shown) in the outflow pathway.

The device 600 also includes integrated electronics (not shown) for implementing the aerosol controller system of the present disclosure, which, in some embodiments, can be programmed with inputted delivery parameters, such as, but not limited to, patient-specific parameters and formulation-specific parameters (e.g., dosing regimen, medications, etc.). The integrated aerosol controller system also records respiration parameters from the air flow sensor and, optionally, waste parameters from a waste sensor, and uses such parameters, in combination with the inputted parameters to calculate delivery parameters and to continuously monitor and optimize delivery parameters. In a specific embodiment, a miniaturized version of the device of the present disclosure will package the computer based, electronic aerosol controller system and electronic controller into a compact assembly that incorporates a microcontroller in place of the computer and data acquisition card. This device will conveniently fit in ventilator circuits for aerosol therapy in patients undergoing mechanical ventilation and will be readily adaptable for use in spontaneously breathing subjects.

Both the hand-held and ventilator-coupled versions can also include various embodiments, such as basic and enhanced versions and those that can accommodate a variety of nebulizers. In an embodiment, a basic version includes internal electronics that detects respiration parameters and that are pre-set to deliver aerosols in synch with the beginning of inspiration and continue for an interval of inspiration. In another embodiment, a more advanced version is able to constantly adapt (a constantly adapting version or CAV) and can be used to further optimize delivery and minimize waste by monitoring of breathing parameters.

A number of nebulizers are commercially available that can be used with the device of the present disclosure. These nebulizers include jet nebulizers that use compressed air to generate aerosols and also “vibrating mesh” nebulizers that contain a porous mesh that vibrates upon electrical excitation to generate aerosols by a micro-pumping action. Both jet and vibrating mesh nebulizers have been tested with embodiments of devices of the present disclosure and have demonstrated that controlled aerosol delivery using the device of the present disclosure enhances delivery with both nebulizer types. Vibrating mesh nebulizers are advantageous in that they tend to be gentler on DNA- and protein-based formulations, and also probably pulmonary surfactant formulations, when compared to jet nebulizers and to minimize waste due to the substantially reduced sputter volume. Further, the vibrating mesh nebulizers introduce essentially no extra air volume into the airway.

In some embodiments of the systems of the present disclosure described above, the respiration sensor includes a dedicated flow sensor to provide a robust method for monitoring patient breathing. The flow sensor provides feedback to the computer system/aerosol controller system in much the same manner as pressure switches. It allows calculation of the volume of air traveling through the ventilator circuit and provides a method to know the volume of air that is generated after aerosol generation is halted. This is particularly important as different amounts of air may be required to chase the generated aerosols to most effectively target select areas in the lung airways.

As discussed above, embodiments of the system/device of the present disclosure include a monitor of exhaled air and waste to allow minimization of waste while still maximizing aerosol delivery to the lungs. This monitor is generally located in/on the expiratory limb of a ventilator circuit or expiratory outlet attached to a mouthpiece connected to a hand-held MDI. The monitor is used to detect waste particles that pass by. Once a threshold is exceeded, the monitor sends an electrical signal to the aerosol controller system informing the system that aerosol delivery parameters should be automatically adjusted to again return the particle counts below the threshold level. Regardless of airflow conditions in the lungs, the device would optimize aerosol delivery and minimize waste.

Delivery can be further enhanced by utilizing the device of the present disclosure to identify airflow conditions that suggest alterations to optimize delivery. The device of the present disclosure is able to associate airflow conditions with a particular clinical scenario and provide recommendations to the hospital staff suggesting alterations to the ventilatory program such as the use of different driving gases and the use of positive end expiratory pressure (PEEP) to optimize delivery.

Chase volume (as discussed above) and gas velocity are important determinants of aerosol delivery to the lungs and can be used by the system of the present disclosure as guides for controlling aerosol delivery. Based on continuous pressure sensing and by also measuring flow in the ventilator circuit, an algorithm/lookup table may be integrated into the device of the present disclosure to maximize deposition and minimize waste based on both patient health and on the physical characteristics of the patient receiving the aerosol.

Software Embodiments

The aerosol controller system of the present disclosure is preferably implemented in software capable of being implemented by a computer system. In embodiments of the aerosol controller software, and in addition to the functional features described above, the software preferably includes features including, but not limited to, the following: tabbed display “pages” including, for instance, a patient data input page for inputting patient-specific parameters such as height, weight, and equipment connection details (e.g., size of intubation tube for ventilated patients, etc.); a formulation-specific page (e.g., which drugs/formulations are being administered, dosage regimen, volume, interactions, etc.); and a real-time data display page for showing current breathing patterns, tidal volume, any drug being administered and amount delivered. The graphical display aspect of the aerosol control software also preferably includes (on the above-described tabbed pages, or other display arrangement) the display of breathing pattern data such as tidal volume, breaths per minute, airflow velocity and/or pressure, and average tidal volumes (for a certain number of breath cycles); the timing during the respiratory cycle in which aerosol generation is occurring; the calculated effective dose; estimated time to complete dosage; waste data; and a plot of historical data for a particular patient.

In addition, the aerosol controller software system preferably includes the ability to control more than one nebulizer at the same time having more than one different protocol. It also preferably includes indicators (e.g., lights, alarm, flashing signal, etc.) for delivering various messages (depending on if it is adapted for ventilator or hand-held use) such as, when nebulizer is active, when to begin treatment, when treatment is complete, when to begin administration, to adjust inhalation velocity/volume, etc. In embodiments, the aerosol controller system can also generate reports with respect to the particular formulations delivered including information such as what was given, how much was given, the calculated effective dose (e.g., how much of what was given was delivered as opposed to wasted), how long treatment lasted, and the like. The aerosol controller system may also include many additional features commonly included in similar software programs and known to those of skill in the art.

Hand-Held Embodiments

As described above and illustrated in one embodiment in FIGS. 7 and 8A-B, various hand-held embodiments of the device/system of the present disclosure can be used by patients at home and those in a hospital not on a ventilator. In some embodiments, a “pre-set” device will initially measure the patient's breathing parameters and calculate the optimal chase volume based on a look-up table that is used to estimate airway volume and dead space. Advanced versions of the hand-held device also include a waste monitor.

In some embodiments, during treatment, the hand-held device will monitor the flow rate to ensure proper aerosol particle entrainment and alert the patient if the rate is too low or too high via indicator lights, such as light 560 in FIG. 7, described above. After the patient has inspired the appropriate volume, another indicator light (or a different signal from the same indicator light) will signal the patient to stop inspiration, breath hold (if necessary) and exhale. In the hand-held version of the device, it only starts (e.g., initiates aerosol generation) when it detects an inspiration, thus allowing the patient to breathe at a rate and volume comfortable to the patient. Moreover, the patient can temporarily stop treatment and resume later if the patient needs to take a break from treatment or some interruption occurs.

One embodiment of the hand-held version is similar to the ventilator version in that it is capable of continual adaptation and has many of the same features (exhaled waste monitor, air flow monitor, etc.) as the ventilator version described above. The primary difference from the ventilator embodiments is that in conscious, spontaneously breathing patients, the tidal volume may not be a consistent amount. To address this potential problem, this hand-held version can initially examine the “normal” breathing pattern of the informed patient to ascertain an effective treatment tidal volume.

For purposes of illustration only, if the normal breathing pattern is found to have a tidal volume of 300 ml, then the treatment tidal volume can be increased to 450 ml. During treatment the device can monitor flow rate to ensure that it is sufficient to properly entrain the generated aerosol particles. If the patient's flow rate is not sufficient, indicator lights can alert the patient to increase the rate of inspiration. Similarly, if the patient is inspiring too quickly the indicator lights can alert the patient to this problem. After the patient has inspired the appropriate volume, the indicator lights can signal the patient to stop inspiration, breath hold (if necessary) and exhale. With each exhalation, the device can monitor the exhaled medication waste and adjust the duty cycle of aerosol generation to maximize lung deposition.

Another potential method to increase lung deposition is to increase the tidal volume by 50-100 ml if the patient is capable of doing so. Another benefit of the hand-held version of the device of the present disclosure is the ability of the patient to temporarily stop treatment and then resume treatment. Since the initiation of aerosol generation is via an inspiration (air flow) sensor, the device can be set aside while the patient takes a break from the medication process or answers the telephone, etc.

A second, and less-expensive embodiment of the hand-held device is appropriate for those patients that do not require absolute maximal lung percentage deposition. It is similar to the previously described device except there is not an exhaled waste monitor, and the duty cycle can be adjusted according to the tidal volume values and a look-up table, but not according to waste output as in the advanced version. For instance, in one possible example, if the effective treatment tidal volume is found to be 450 ml then the duty cycle could be set to 0-40%, or if the effective tidal volume is found to be only 350 ml then the duty cycle could be decreased to 0-23%.

Similar to the ventilator version, either hand-held embodiment can allow multiple aerosol generators to be inserted to allow multiple drugs to be administered in serial, alternating or simultaneous manners. For the version that monitors exhaled medication waste, the device can calculate the “effectively administered medication dose” and time-stamp the treatment to allow documentation of the patient's treatment compliance. Similar, though less exact, time-stamps can be collected for the simpler version of the hand-held device.

The device can also monitor if the correct medication is being administered; which could be a potential problem for patients that require more than one type of medication via aerosol administration. To document patient compliance for each medication, the device can compare time stamps with the present time to determine if the patient is attempting to administer medication at the appropriate time. If the patient fails to medicate after an appropriate “grace time”, the device can indicate (e.g., with sounds and/or lights) that it is time to start medication. One additional embodiment of the device includes a detachable timer that can be carried by the patient (in the pocket, around the neck, in the purse, etc.) to warn of missed medication. The only way to “silence” the timer is to insert it into the device during the appropriate medication. After finishing the medication, the timer is reset to alarm at the next medication interval.

Embodiments can also include various methods to prevent a patient from administering the wrong medication or administering it at the wrong time. For instance, color coordinated reservoirs for a specific drug can aid in this effort. Additionally or alternatively, the reservoir can be variably shaped and/or have identification markings that the device can identify that indicate the proper dosage, appropriate time for administration, and device parameters that should be used for a particular medication.

In other embodiments, for those occasions in which a drug must be administered as quickly as possible, the mouthpiece (e.g., mouthpiece 550 in FIG. 7 above) can be rotated 180 degrees to initiate continuous aerosol generation. When the mouthpiece is in this position all of the indicator lights will flash to indicate that continuous aerosols are being generated.

In other embodiments, a manually or electrically-adjustable air flow restrictor 630 can be attached to assist a user in achieving the correct inspiratory rate/volume.

Maximal Aerosol Deposition Efficiency

When it is desirable to maximize lung deposition of a given dose of drug, due to cost and/or scarcity, the device of the present disclosure can allow up to about 80-85% or greater to be deposited in the airways. This can be done by monitoring and controlling a number of variables such as, but not limited to, optimizing chase volume, minimizing waste via monitoring, and optimizing particle size. Variations of this process are described in general in the flow charts above, and a detailed, exemplary embodiment is presented below, but the disclosure is not intended to be limited by any particular embodiment.

In an exemplary embodiment, the device of the present disclosure measures the gas flow rate in the inspiratory limb of the ventilator circuit and calculates an averaged effective tidal volume. The nebulizer is actuated at an optimal point during the breath cycle and stops at some point during inspiration to allow the remaining portion of the tidal volume to act as a “chase” volume for the aerosol. Depending on the value of the tidal volume the device of the present disclosure will initially stop aerosol generation at a point (e.g., about 20 percent of the tidal volume) where it is estimated that a minimal amount of exhaled waste aerosol occurs. During this time a sensor on the expiratory limb of the ventilator circuit monitors the number of exhaled aerosol particles and calculates the amount of waste being generated. The 20% “duty cycle” is increased in steps of about 5% (or some other percent) until the monitor measures a significant increase in exhaled waste and then is decreased to the previous duty cycle.

At this point the duty cycle is increased in smaller steps (e.g., about 1%) until the maximal duty cycle with the minimal amount of exhaled waste is ascertained. At each step increase, multiple breath cycles (e.g., 10 breath cycles) are allowed to occur but the measured waste may be averaged for only a certain number of the breaths (e.g., the final 5 breaths). During drug administration, the device of the present disclosure will continuously monitor exhaled waste and after a certain number of breaths (e.g., ten breaths), it will readjust the duty cycle (increase and/or decrease by 1% or other % as appropriate) to maintain maximal deposition rate with minimal exhaled waste aerosol.

The size of the generated aerosol particles can have a significant effect on the degree of drug deposition. Extremely small particles are less likely to deposit in the lungs and will be exhaled, whereas large particles will be more likely to decrease deposition by an increase in the amount of aerosol “rainout” waste, as defined above.

Aerosol Deposition at Specific Airway Sections

The methods described above for maximal aerosol deposition efficiency are most effective for distal airway deposition. If larger airway deposition is desired, this can be achieved by initiating aerosol generation later in the inspiratory pathway and/or by increasing the size of the aerosol particles. A hypothetical example to cause deposition in the larger airways would be to increase the size of the aerosol particles from 2 to 4 microns and initiate nebulization at 30% of tidal volume and stop at 70%. The aerosol controller system of the present disclosure can automatically select the optimal aerosol particle size and delivery timing for an inputted delivery parameter specifying a particular target airway location.

Adjustable Efficiency Embodiments

Embodiments for maximal aerosol deposition efficiency were discussed above, for achieving the maximum airway deposition with the minimum amount of waste. However, it can take longer to deliver a formulation to a patient at such high efficiency. Alternatively, there may be situations that require administering drugs via aerosol as rapidly as possible, while still minimizing waste, and this can also be achieved with the device of the present disclosure. A nebulizer can be set to initiate aerosol generation prior to inspiration thus generating a “charge volume” while extending the duty cycle up to the end of inspiration. After administering the “loading dose” the device of the present disclosure can then be set to give a precise “maintenance dose” as described above. If a second drug is being administered during the loading dose of the first drug, the device of the present disclosure can either maintain its settings for maximal deposition or default to a setting that is historically effective for precise drug administration; for example, a duty cycle of 40% of the tidal volume initiated at the start of inspiration.

Sometimes, it may be desirable to deliver a formulation at high efficiency during one administration (or for a particular drug), but desirable to deliver a formulation more rapidly, with less concern for efficiency, in another administration (or for a different drug). Thus, embodiments of the systems, devices, and methods of the present disclosure include the ability to adjust efficiency or the “effectiveness” setting, such as by including an efficiency controller (e.g., an efficiency or effectiveness dial or knob).

In one embodiment, the system has an adjustable efficiency controller in which the efficiency setting that can be set to the highest efficiency with minimal waste (e.g., starts 0.25 seconds after start of inspiration and stops once 30% of tidal volume has been given), set to relatively lower efficiency at continuous nebulization, or set somewhere in between the highest and lowest setting. In embodiments, the operator must reaffirm the setting if either of the above extremes are to be used.

In an illustrative example of an embodiment having a variable efficiency setting, a device of the present disclosure has an effectiveness knob with a certain number (e.g., 21) of settings (e.g., 0 to 100 at intervals of 5). In this example, 100 represents the most efficient relative to dose, and 0 represents the most rapid delivery with acceptable waste tolerance. Various settings on the effectiveness knob are shown in Table 1 below, where the 40% is calculated from dead space and tidal volume so the actual value would vary from patient to patient.

TABLE 1

| | |
|---|---|
| 100% | Starts in synch with inspiration initiation |
| | and stops at 40% of inspiration |
| 95% | Starts at −0.25 s before inspiration begins and stops at 40% |
| 90% | Starts at −0.5 s before inspiration begins and stops at 40% |
| 85% | Starts at −0.75 s before inspiration begins and stops at 40% |
| 80% | Starts at −1.0 s before inspiration begins and stops at 40% |
| 75% | Starts at −1.25 s before inspiration begins and stops at 40% |
| 70% | Starts at −1.5 s before inspiration begins and stops at 40% |
| 0% | Starts at end of inspiration and stops at point where chase |
| | volume equals deadspace. |

The measured tidal volumes and respiratory rate for a particular patient will then allow calculation of when the nebulization will start and stop via empirical equations. Using this information and the effectiveness setting, the system can calculate how much drug is needed in the reservoir for the person to receive the drug dose desired. It can also calculate how long the treatment should last.

In another exemplary embodiment of a system/device according to the present disclosure having an effectiveness dial, the dial may have several dial positions allowing emphasis on delivery time and/or efficiency. In a particular example the dial has 23 positions as shown below in Table 2, with dial position 1 (number 0 in table) emphasizing rapid delivery, and dial position 23 (number 22 in table) providing for most efficient delivery. In use, the aerosol controller system first calculates inspiration (I) and expiration (E) times based on acquired data/respiration parameters. The system then calculates aerosol laden inspiration volume (ALIV) with the following equation: ALIV(ml)=Tidal Volume (TV)−Dead Space.

TABLE 2

| | |
|---|---|
| 0 | Start nebulization at end of E and stop when ALIV has been given |
| 1 | Start at time −100% E relative to inspiration initiation and stops at Y |
| 2 | Start at time −95% E relative to inspiration initiation and stops at Y |
| 3 | Start at time −90% E relative to inspiration initiation and stops at Y |
| 4 | Start at time −85% E relative to inspiration initiation and stops at |
| 5 | Start at time −80% E relative to inspiration initiation and stops at Y |
| 6 | Start at time −75% E relative to inspiration initiation and stops at Y |
| 7 | Start at time −70% E relative to inspiration initiation and stops at Y |
| 8 | Start at time −65% E relative to inspiration initiation and stops at Y |
| 9 | Start at time −60% E relative to inspiration initiation and stops at Y |
| 10 | Start at time −55% E relative to inspiration initiation and stops at Y |
| 11 | Start at time −50% E relative to inspiration initiation and stops at Y |
| 12 | Start at time −45% E relative to inspiration initiation and stops at Y |
| 13 | Start at time −40% E relative to inspiration initiation and stops at Y |
| 14 | Start at time −35% E relative to inspiration initiation and stops at Y |
| 15 | Start at time −30% E relative to inspiration initiation and stops at Y |
| 16 | Start at time −25% E relative to inspiration initiation and stops at Y |
| 17 | Start at time −20% E relative to inspiration initiation and stops at Y |
| 18 | Start at time −15% E relative to inspiration initiation and stops at Y |
| 19 | Start at time −10% E relative to inspiration initiation and stops at Y |
| 20 | Start at time −5% E relative to inspiration initiation and stops at Y |
| 21 | Start at time 0% E relative to inspiration initiation and stops at Y |
| 22 | Start at time 5% TV relative to inspiration initiation and |
| | stops at 30% TV |

Y = Factor * ALIV; Factor ranges from 0.01-0.99

The embodiments described above are merely exemplary, and it will be understood by one of skill in the art that an effectiveness/efficiency dial/knob can be designed any number of ways with any number of different settings, and such embodiments are intended to be included in the present disclosure.

Additional Embodiments and Advantages

There are several attributes of the device of the present disclosure that can also benefit aerosol drug therapy. One is the ability to generate aerosols at selected intervals; e.g., 10 aerosol cycles at 15 minute intervals. Another attribute is the ability to use multiple aerosol generators to administer different drugs at selected intervals. These multiple drugs can be administered simultaneously, consecutively, or in an alternating manner. One hypothetical example would be the desirability to give a bronchial dilator to allow better deposition of a second drug. The flexibility of the device of the present disclosure will allow a drug to be administered at variable time points while second or third drugs are administered at time intervals deemed to be most effective for that particular drug. By monitoring exhaled waste, more precise dosing of a drug can be achieved to prevent either under or overdosing of the patient.

In some embodiments of the present disclosure, low density gases can be used in the ventilator circuit to enhance airflow to constricted areas in the lungs and to reduce turbulent flow, thus decreasing loss of aerosol in ventilator tubing and endotracheal tube and enhancing deposition in the lower respiratory tract. Flow sensors integrated into the device of the present disclosure can be compatible with different gases that can be tailored to specific patient needs.

Now, having described the systems, devices, and methods of controlling and delivering aerosol formulations of the present disclosure in general, the following exemplary embodiments are provided. The specific examples below are to be construed as merely illustrative, and not limitative of the remainder of the disclosure in any way whatsoever.

### EXAMPLES

### Example 1

Controlled Aerosol Delivery in a Ventilator Circuit Reduces Waste and Enhances Deposition In Vitro.

The In vitro Testing Apparatus: The following in vitro studies were designed to assess the effects of nebulization timing and duration on the efficiency of radioisotope aerosol deposition and the magnitude of the wasted fraction in a ventilator circuit. As illustrated in FIG. 9, corrugated tubing was connected to the inflow and outflow ports of an Ohmeda anesthesia ventilator machine, and oxygen-enriched air was passed through the system at a tidal volume of 500 ml (˜7 ml/kg based on a 70 kg human). Differential pressure sensors (World Magnetics, Traverse City, Mich.) were combined with an airflow resistance coupler and placed in the inspiratory and expiratory limbs of the ventilator circuit. Signals from each sensor were sent with each ventilator cycle to an electronic controller/data acquisition system (computer with DAQCard 1200 PCMCIA card and a Labview software interface and used to define the timing and duration of inspiration and expiration. A historical accounting of each breath trace was logged in the system memory and information from each previous breath trace used to provide a basis for timing of aerosol generation at select intervals during ventilation, based on input into the software graphical user interface (GUI).

The experiments allowed measurement of the mass of aerosols deposited in a HEPA filter (“lung” filter) positioned at the end of an endotracheal tube immediately upstream of a compliant balloon and the mass of aerosols deposited in HEPA filters placed in the exhalation limb of the ventilator circuit. The total waste is determined by rainout, “wrap-around”, exhaled waste, and sputter volume as defined above. The mass of aerosols deposited on the waste filter is a sum of the “wrap-around” and exhalation wastes only.

Studies with Vibrating Mesh Nebulizer: An essentially zero flow vibrating mesh nebulizer was placed (Aeroneb Pro from Aerogen, Mountain View, Calif.) in the inspiratory limb of the ventilator circuit diagrammed in FIG. 9. A 0.5 ml dose of ^{99m}Tc-DTPA radiotracer was placed in the nebulizer and delivered into the circuit. The effects of controlled aerosol delivery on the amount of isotope that deposited in a HEPA filter situated upstream of a compliant balloon used to simulate a lung and the amount of isotope waste collected in a second HEPA filter situated 6 cm from the y-piece split were determined. Aerosols were generated in 0.75, 1.125, 1.5, or 3.0 second intervals corresponding to 25, 37.5, 50, and 100%, respectively, of the 3 seconds allowed for inspiration until the nebulizer was dry. For each of these time durations, aerosol was generated beginning either 0.5 seconds before inspiration, coincident with the initiation of inspiration, or at 0.5, 1.0, 1.5, or 2.0 seconds after the beginning of inspiration and continued for the times indicated above. Control studies in which aerosol was generated throughout the respirator cycle as is usual for standard nebulizers were also conducted. These studies allowed examination of the effects of aerosol generation timing vis-à-vis the respiratory cycle, duration, and the subsequent effects of chase volume on deposition efficiency.

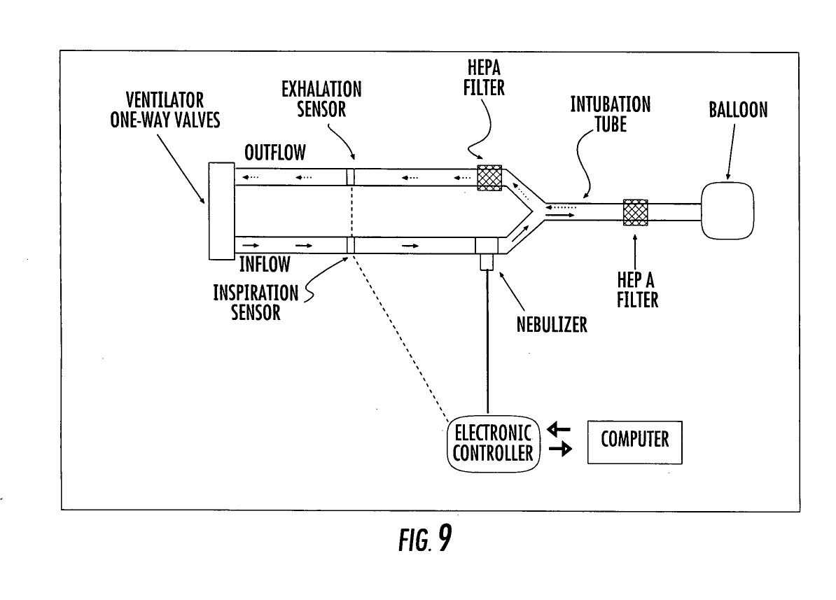

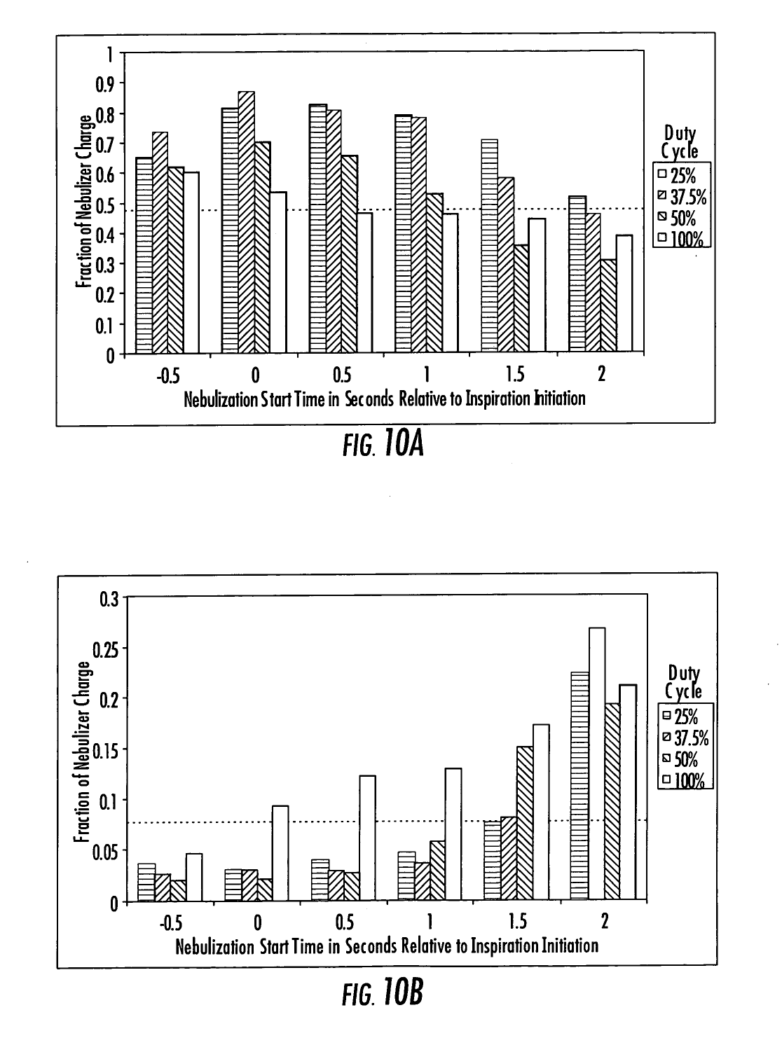

As indicated in FIG. 10A, aerosol deposition in the “lung” filter was maximal when aerosol generation was initiated during the early phases of inspiration (reference legend). Aerosol deposition was enhanced under conditions when the time of aerosol generation was 0.75 seconds (25% of inspiration) or 1.125 seconds (37.5% of inspiration) when compared to aerosol generation for 50% or 100% of inspiration. Controlled aerosol delivery under nearly all conditions where aerosol generation was initiated between −0.5 and 1 second relative to the beginning of inspiration resulted in substantially higher “lung” deposition efficiencies than when aerosols were generated continuously (see horizontal dashed line corresponding to the continuous aerosolization conditions in FIG. 10A). Lung deposition was enhanced even when aerosol production was not initiated until 1.5 seconds after the start of inspiration, so long as aerosols were generated for 25-37.5% of inspiration. The drop in lung deposition when generating aerosols for longer periods of time during inspiration is likely due to an insufficient chase volume to clear aerosol from the airway dead space into the distal lung. Deposition of the waste filter is shown in FIG. 10B. When these data were expressed as a lung/waste count ratio (not shown), the use of the device of the present disclosure resulted in as much as a 5-fold improvement in aerosol efficiency when compared to the continuous aerosolization condition.

Since results were similar when aerosol was generated for 25% and 37.5% of inspiration, and since pulsing for 37.5% of inspiration allows faster delivery of a given volume, additional studies were conducted with aerosol durations of 37.5% of inspiration to determine effects of the time relative to inspiration at which aerosol generation was initiated. Consistent with the initial studies at 37.5% duty cycle, aerosol deposition was maximized when aerosols were started coincident with the beginning of inspiration and stopped after 1.125 seconds thus providing an ˜331 ml chase volume (FIG. 11). Lower levels of deposition when aerosols were started 0.5 seconds before inspiration were a result (via visual observation) of non-entrained aerosol (e.g., rainout). When aerosol generation was not started until 1.5-2 seconds into inspiration, decreased deposition was due to a decreased chase volume (with the negative value for chase volume indicating that aerosol generation was not stopped until the exhalation phase had already begun). In other words, at these points, the aerosols are generated so late that the dead space volume of the system remains filled with aerosols at the end of inspiration. Coincident with the decreased fractional deposition in the “lung” filter when initiating aerosol generation later during inspiration, the amount of aerosol collected in the waste filter increased. These data suggest that aerosol delivery can be optimized in ventilated patients and deposition markedly improved with controlled aerosol delivery using the device of the present disclosure. Further, the chase volume observations suggest that an externally placed sensor for detecting exhaled aerosols could be used to provide a feedback signal to the device of the present disclosure as a basis for adjusting delivery parameters to minimize exhaled waste. This feature would automatically adjust the aerosol delivery pattern to the patient compensating for differing dead space volumes.

Studies with Jet Nebulizers: Studies were also conducted to determine whether the use of the device of the present disclosure to control aerosol delivery from a jet nebulizer would enhance aerosol deposition efficiency in the above-described in vitro setup. The data is shown in FIG. 12. A Circulaire® jet nebulizer (Westmed) without the rebreathing bag was loaded with 1.5 ml of ^{99m}Tc-DTPA in saline, and the aerosols were generated continuously using compressed air at 50 psi and at a flow of 8 L/min until nothing remained in the nebulizer but the sputter volume. Then 1.5 ml aliquots of the ^{99m}Tc-DTPA solution were subsequently loaded and aerosols generated with the device of the present disclosure set to deliver aerosol during selected portions of inspiration as described above (with an n=2 for each delivery routine). Results were similar to those for the Aeroneb Pro nebulizer. When aerosols were generated with the device of the present disclosure, aerosol delivery to the “lung” filter was substantially increased and the amount of “wasted” aerosol was substantially reduced compared to continuous aerosol generation. These results indicate that the device of the present disclosure will enhance aerosol delivery efficiency with either vibrating mesh or jet nebulizers.

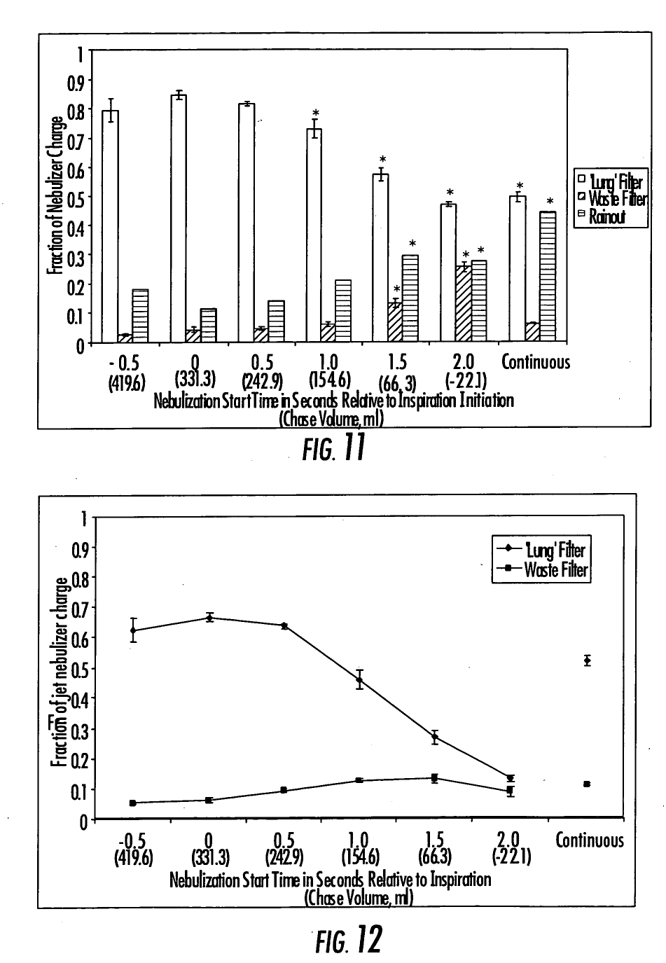

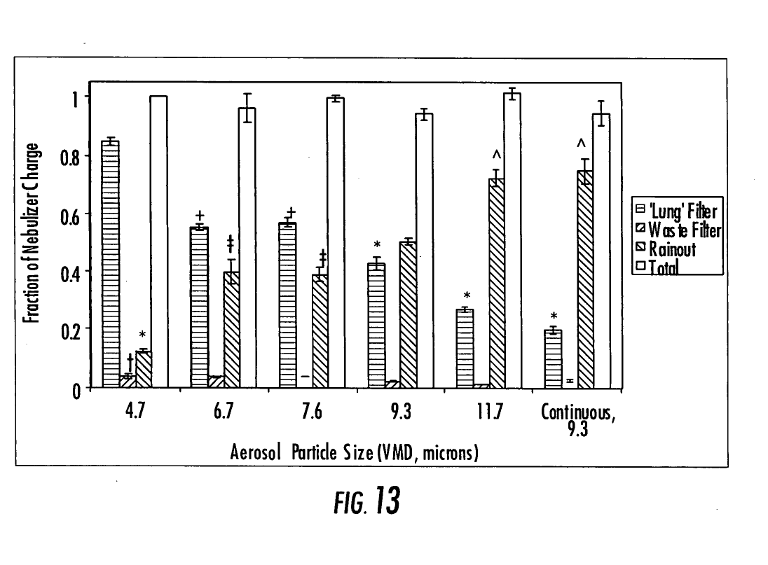

Studies on particle size and distribution: As targeting select areas in the lungs is a goal of the present disclosure, the effects of aerosol particle size on deposition was also tested in vitro, as was the utility of the device of the present disclosure in enhancing delivery even for larger size aerosols (FIG. 13). Briefly, the commercially available Aeroneb Pro (4.7 μm volume mean diameter, VMD) and four additional vibrating mesh nebulizers that were special prototypes containing membrane pores of sizes to produce aerosols ranging from 6.7-11.7 μm VMD were provided by Aerogen. Aerosols were generated in synch with inspiration and continued for 1.125 seconds and collected in the “lung” and waste filters as discussed above. The ventilator circuit tubing from the nebulizer to the y-piece was wiped after each delivery, and the activity in the “swabs” was counted to provide a measure of rainout waste. Maximal “lung” filter deposition was obtained for the 4.7 μm nebulizer with the fraction of delivered dose decreasing with increasing pore size. The reductions in “lung” filter activity were associated with concomitant substantial increases in rainout waste due to the larger aerosols that were generated. The amount of isotope collected in the waste filter actually decreased with increasing pore size probably due to less isotope reaching the y-piece due to increased rainout. Mass balance occurred in all cases (e.g., the activity loaded into each nebulizer was completely accounted for by measurements of the in vitro “lung” filter, waste filter, and the swabs).

In some circumstances, it might be desirable to deliver larger aerosols while minimizing sources of waste. Approximately 43-57% of the aerosols generated with the nebulizers with the three intermediate size pores were collected in the “lung” filter. Though the described in vitro setup does not simulate aerosol delivery through the lung airways, this 43-57% represents a significant fraction of larger size aerosols reaching the “lung” filter, which ultimately might allow targeting of different areas of the lungs. To determine if the use of the device of the present disclosure enhanced delivery of larger size aerosols, additional studies were conducted using the 9.3 μm-aerosol producing nebulizer in which results following continuous nebulization were compared to those obtained with this same nebulizer under control with the device of the present disclosure. As illustrated in FIG. 13, the use of the device of the present disclosure resulted in a 2-fold increase in “lung” filter counts and a ⅓ reduction in rainout waste. These studies indicate that controlled aerosol delivery might be useful in enhancing delivery of larger aerosols for targeting the central airways.

Studies on inspiratory:expiratory ratios and particle size on delivery efficiency: To test the effects of aerosol delivery at different inspiratory:expiratory ratios (I:E), the test lung (FIG. 9) was ventilated at a 1:1 or 1:3 I:E ratio. Radioactive saline aerosols containing ^{99m}Tc-DTPA were generated with the 4.7 and 9.3 μm-aerosol producing nebulizers operated continuously and during phasic delivery starting in synch with inspiration and continuing for a 37.5% duty cycle (e.g., 1.125 seconds of 3 second inspiration). Isotope collected in the test lung and waste filters was counted and results normalized to the amount of isotope initially placed in the nebulizer. The results are illustrated in FIG. 14.

### Example 2

Compare Performance of the Device of the Present Disclosure with Conventional Continuous Aerosol Delivery in Mechanically Ventilated Healthy Sheep and Sheep with Acute Lung Injury.

In anesthetized sheep with either normal lung mechanics or during bronchoconstriction, the effects of phasic aerosol delivery on the amount of radioisotope deposited in the lungs and the amount expired (as collected in a waste filter) were tested. Results were compared to those in which aerosol was delivered continuously. Sheep were anesthetized and ventilated with oxygen-enriched air at a tidal volume of 500 ml and a respiratory rate of 10 breaths per minute. The ventilator setup is illustrated schematically in FIG. 15.

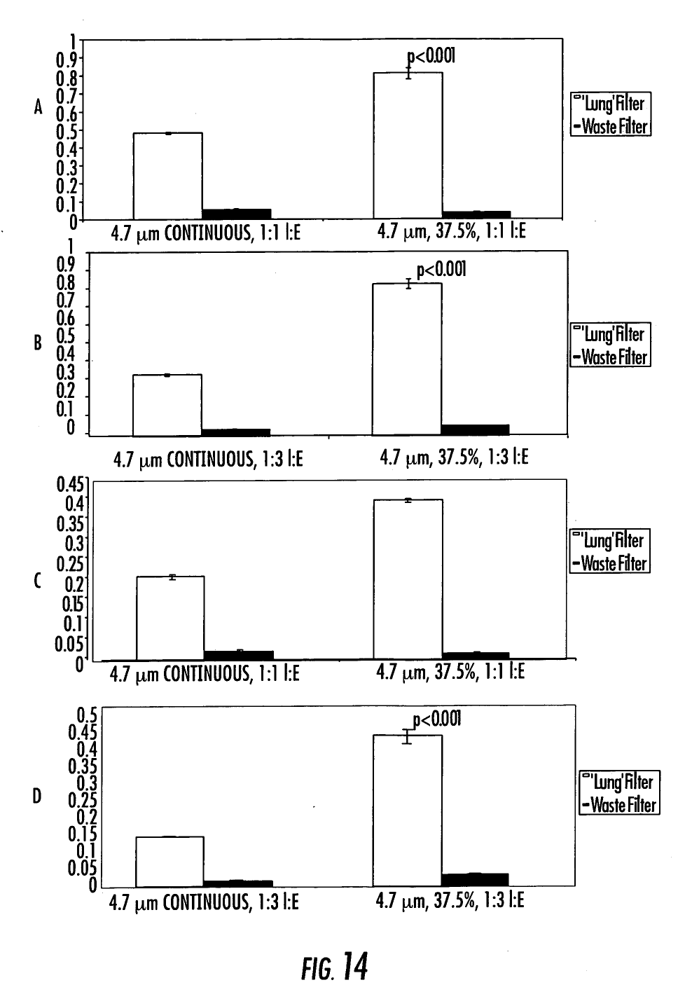

A lead-shielded gamma scintillation probe (Bicron 2M/2, Saint Gobain Crystals and Detectors, Newbury, Ohio) was placed on the chest wall, and gamma emissions from a subjacent portion of lung monitored through an isotope detection system that included a multichannel analyzer board (Model ASA-100, Can berra Industries, Meriden, Conn.) and software interface (Gamma Analysis option of Genie 2000 software, Can berra Industries). After a baseline period, 0.5 ml of ^{99m}Tc-DTPA was added to a vibrating mesh nebulizer, and aerosol generation was initiated at the times described in the preceding section. Between each time adjustment, the waste collection filter was replaced with a non-radioactive filter and the study continued. ^{99m}Tc activity was also calculated from measures made at three minute intervals immediately after deposition to correct for DTPA clearance. As indicated by the blue bars in FIG. 16 and consistent with the in vitro studies, aerosol generation started either 0.5 seconds before the beginning of inspiration or coincident with beginning of inspiration and continued for 1.125 seconds (37.5% of inspiration volume) resulted in significantly higher lung deposition than aerosol generation initiated later during inspiration (with lower chase volume) or when nebulization was continuous. Since a single gamma scintillation probe to assess lung deposition, only a portion of the lung tissue was actually “seen” by the probe. Thus, in FIG. 16, the lung counts are normalized to peak activity in the lungs of each animal.

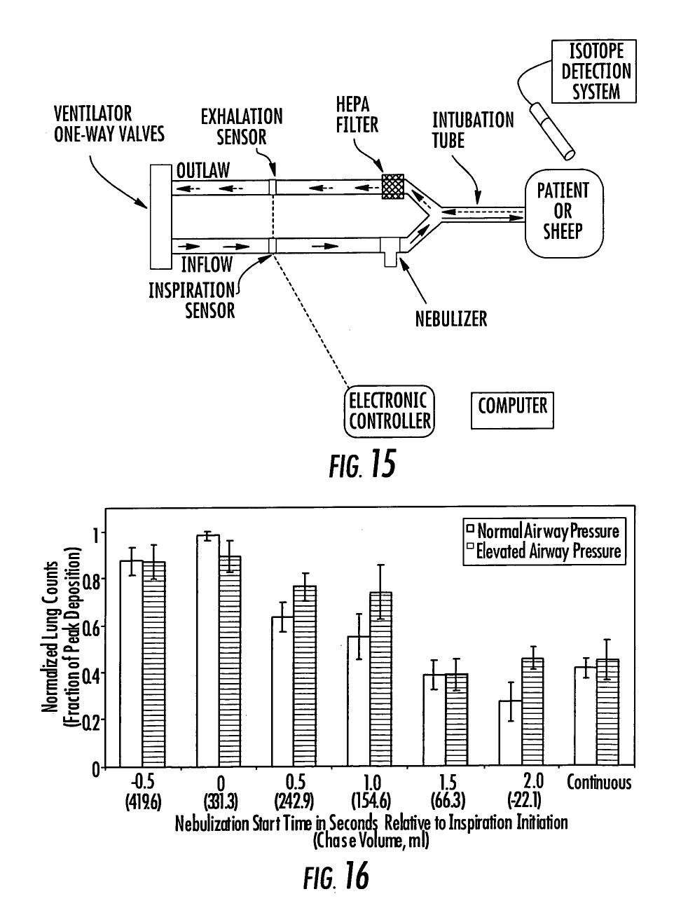

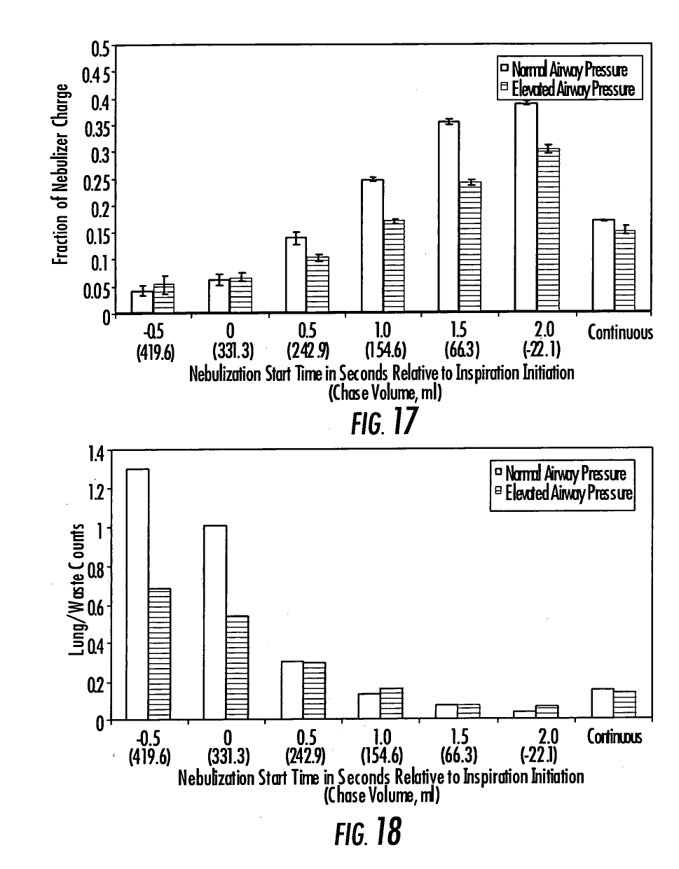

At the end of the study, the activity in the waste filters was measured using the same isotope detection system described above. As shown in FIG. 17, waste collected during controlled aerosol delivery with the device of the present disclosure was at a minimum when aerosol generation was started 0.5 seconds before or coincident with the start of inspiration. Waste progressively increased as aerosol generation was initiated later into inspiration, a pattern similar to that observed in vitro as discussed above. The ratio of the lung counts to waste counts demonstrated substantial improvement in aerosol delivery efficiency with phasic delivery controlled by the device of the present disclosure, particularly when aerosol generation was initiated just before or early in inspiration (FIG. 18).

FIGS. 16-18 also illustrate effects of bronchoconstriction produced by an aerosol of sodium arachidonate on aerosol delivery efficiency. These studies were conducted after the control studies discussed above. Arachidonic acid was converted to sodium arachidonate by the procedure of Ogletree and Brigham and was aerosolized (Dose=1.5 grams/50 ml typically diluted 1:3) to cause marked bronchoconstriction and/or decreased lung compliance and thus elevated airway pressure. During bronchoconstriction, airway pressure increased to 48-50 cm H₂O within 20 minutes and was associated with marked hypoxemia. During bronchoconstriction, ^{99m}Tc-DTPA was administered by phasic aerosol delivery while maintaining the arachidonate delivery. As illustrated in FIG. 16, bronchoconstriction resulted in a decrease in lung deposition. However, phasic aerosol delivery with generation initiated within ±0.5 or +1 second relative to the beginning of inspiration (thus allowing chase volumes >˜155 ml) improved aerosol deposition when compared to the continuous aerosolization control; the effect was less than that observed under normal airway conditions.

Similar deposition levels were achieved under normal conditions and during bronchoconstriction except when aerosols were generated either immediately before or coincident with initiation of inspiration. This was probably because of increased turbulent flow during bronchoconstriction resulting in aerosol deposition in areas outside the field of view of the probe. The amount of waste was lower with phasic delivery initiated in the early stages of inspiration (FIG. 17). The increase in waste when initiating aerosol delivery at the later time points was associated with a decreased chase volume. Normalizing the lung counts to those found in the waste filter revealed a substantial improvement in aerosol deposition efficiency when aerosol generation was initiated immediately before or coincident with initiation of inspiration (FIG. 18).

When compared to continuous aerosol delivery, efficiency of deposition could be increased nearly 9-fold under normal airway pressure conditions and 4-5 fold during bronchoconstriction. These data suggest that controlled aerosol delivery with the device of the present disclosure substantially improves aerosol deposition efficiency even during marked bronchoconstriction. The use of less dense ventilation gases may enhance this effect even further.

### Example 3

Radioaerosol Waste is Enhanced Following Endotoxin-Induced Acute Lung Injury

This study was designed to compare the wasted fraction of ^{99m}Tc-DTPA aerosols delivered continuously with the zero flow nebulizer to control sheep (n=6) and sheep administered 2 mg/kg E. coli endotoxin. This dose of endotoxin in sheep causes a period of intense bronchoconstriction and pulmonary vasoconstriction followed by pulmonary edema. The experimental setup was similar to that illustrated in FIG. 15, except that no scintillation probe was used and isotope waste was measured by counting radioactivity in a filter placed in the expiratory portion of the ventilator tubing using a gamma energy counter. The wasted fraction of aerosol was more than 3-fold higher during endotoxin-induced lung injury with endotoxin [3,836,141 (n=1)] than when the lungs were normal [1,289,102±125,326 (n=6)]. These results and those of Example 2, above, suggest that the use of the device of the present disclosure and positive end expiratory pressure (PEEP) during acute lung injury will significantly reduce waste and enhance aerosol deposition in the lungs.

### Example 4

Controlled Aerosol Delivery Effectively Targets the Lung Airways

The present study was designed to assess aerosol deposition patterns using jet nebulizers and the device of the present disclosure. These studies were conducted in the Vanderbilt University Medical Center Hawkeye scanning facility. Each sheep was surgically instrumented with catheters in a jugular vein and a carotid artery. After allowing several days for the sheep to recuperate from surgery, they were anesthetized, intubated, ventilated, and placed on the GE Hawkeye scanner bed. After a 30 minute acclimatization period, 500 μCi of ^{99m}Tc-DTPA and 250 μCi of 131I-albumin were added to a syringe and the volume brought to 5 ml with normal saline. The contents of the syringe were then added to a nebulizer and aerosols generated for 30 minutes during ventilator-assisted inspiration. Planar images were taken every two minutes with anterior and posterior cameras. Nebulization was then halted, and clearance of the radiotracers over two hours was measured with Hawkeye in an identical manner. At the end of the radioisotope clearance image acquisition, a rotational CT image was taken of the sheep thoracic cavity to delineate lung boundaries and chest anatomy. The sheep was sacrificed and its lungs excised. Lungs were placed on grid paper and diced with a rectangular cutting grid. Lung tissue was placed in plastic vials and gamma energy emissions of each isotope measured with a gamma counter.

When the Hi-Flo MiniHeart nebulizer was used, deposition profiles were clear and there was an obvious accumulation of both the ^{99m}Tc-DTPA and ¹³¹I-albumin during aerosol generation (FIG. 19, composite of ^{99m}Tc-DTPA and ¹³¹I-albumin deposition). Surprisingly, much of the tracer remained at 2 hours after halting aerosol generation (data not shown). When the Neb 3A+ nebulizer was used, accumulation of the radioisotopes in the lungs was evident (data not shown) but the signal was considerably less. This was probably because the output of the Neb 3A+ is only ˜½ that of the Hi-Flo MiniHeart®. More radiotracer was not used with the Neb 3A+ in order to avoid anything that would affect aerosol size other than the choice of nebulizer.

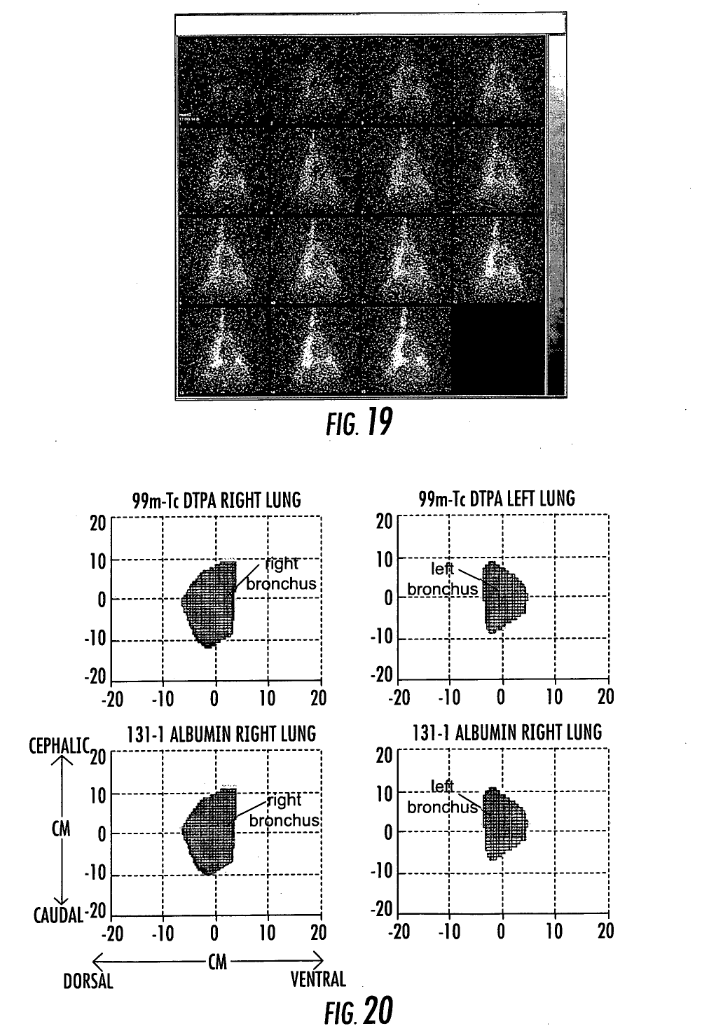

As illustrated in FIG. 20, radioisotope distributions in lung tissue (as assessed from gamma energy emissions counting of lung tissue samples) revealed a higher dorsal (towards back) and caudal (towards tail) deposition of both the ^{99m}Tc-DTPA and ¹³¹I-albumin. These areas anatomically correspond to predominantly smaller airways and would indicate that the Hi-Flo MiniHeart® nebulizer deposits aerosols distally.

### Example 5

DNA Formulated with Liposomes Maintains Supercoiled Form after Aerosolization with the Aeroneb Pro Nebulizer

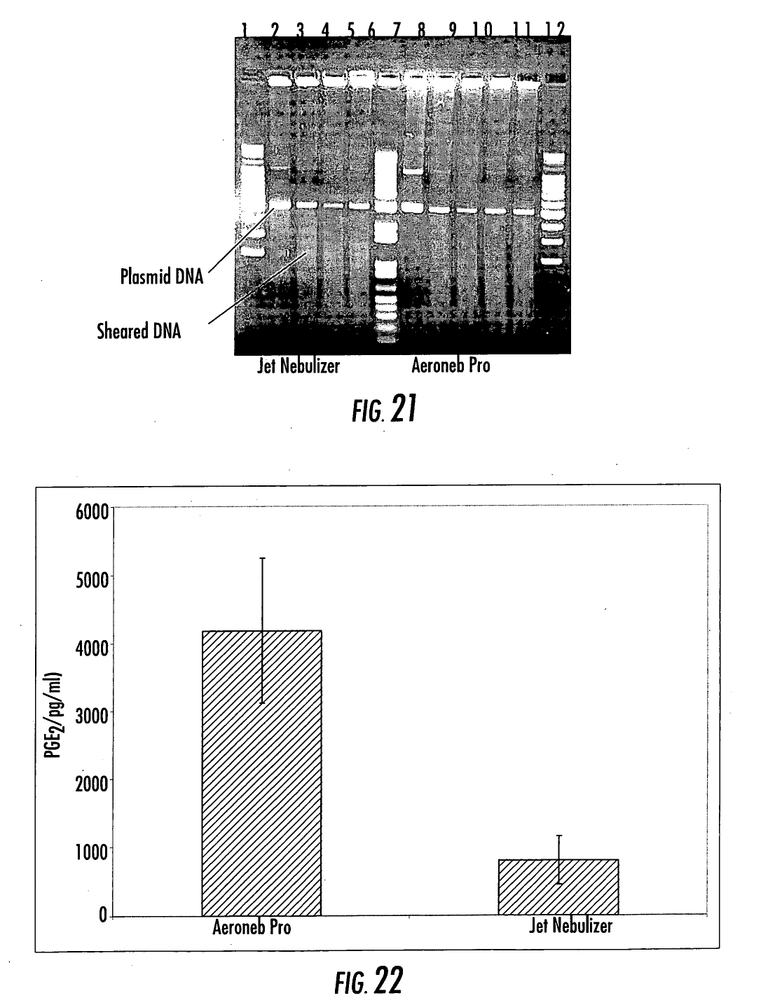

The present example examined the effects of aerosolization of a DNA-based lipoplex formulation on DNA integrity. Briefly, liposome and plasmid were formulated at a 3:1 w:w lipid:DNA ratio and aerosolized using either a jet nebulizer or a zero flow vibrating mesh nebulizer (Aeroneb Pro, Aerogen, Mountain View, Calif.). Normally at a 3:1 w:w ratio, about 20% of the plasmid is not bound to liposome. As shown in FIG. 21, the integrity of DNA after 5, 10, and 12.5 minutes of aerosolization (corresponding to lanes 8, 9, and 10 respectively) was mostly maintained as shown by the ˜5,200 bp bands (plasmid DNA). There was some breakdown of product as illustrated with the slight smearing below the plasmid bands. Unaerosolized formulation is shown in lane 7, and an aliquot of the lipoplex remaining in the nebulizer after 12.5 minutes is shown in lane 11. Similar measures following aerosolization with a jet nebulizer exhibited a more pronounced degradation in the integrity of the DNA as demonstrated by the brighter smeared areas below the ˜5,200 bp bands in lanes 2-5 (sheared DNA), which correspond to aerosols generated for 5, 10, or 12.5 minutes (lanes 2, 3, and 4 respectively) and that remaining in the nebulizer after the 12.5 minutes (lane 5). All other lanes represent DNA ladders.

Potency of a Gene-Based Therapeutic is Maintained after Aerosolization with the Aeroneb Pro Nebulizer

One of the traditional difficulties with aerosolizing liposome-DNA complexes has been identifying nebulizers that have a high output and do not compromise the potency and integrity of the lipoplex formulation. As described earlier, recent advances in aerosol technology include the use of vibrating meshes through which drug solution is pumped through micro-pores to generate aerosols with minimal generation of volume. One of these nebulizers was used to examine the effects of nebulization on emitted dose and the potency of one of the geneRx+ lead gene-based products (a plasmid containing a gene encoding human cyclooxygenase I complexed with cationic liposomes, referred to as Coxagen). 10 ml of lipoplex formulation was prepared including composed of sonicated DOTAP:DOPE liposomes complexed with plasmid in a 3:1 lipid/DNA ratio.

Then 4.5 ml of the formulation was placed into a standard jet nebulizer or into the vibrating mesh-based Aeroneb Pro nebulizer. Aerosols were generated using compressed air at 10 L/min for the jet nebulizer or using the vibrating mesh technology in the Aeroneb device. Aerosols were collected in cooled tubes by using an external vacuum source to draw in the aerosol when using the jet nebulizer or directly into the cooled tube when using the vibrating mesh-based device. The aerosols were generated for 12.5 minutes (the time necessary to completely nebulize the 4.5 ml solution) and collected at 5, 10, and 12.5 minutes of nebulization.

The quantity of DNA in each vial was determined by measuring DNA concentration in a spectrophotometer after dissolving away the liposomal component (which interferes with the optical density reading). Cultures of a normal human bronchial epithelial cell transformed cell line (BEAS) were then transfected with equivalent amounts of product based on equal amounts of DNA. The transfection mixture was allowed to incubate with the cells for 4 hours, at which point the mixture was removed and the cells incubated for an additional 20 hours in media. The media was removed at 24 hours, and fresh media containing 40 μM arachidonic acid was added for 1 hour. The media was collected and assayed by EIA kits from Cayman for PGE₂ (the principal prostanoid produced by these cells).

Neither nebulizer showed an effect of duration of nebulization on PGE₂ expression (data not shown). As illustrated in FIG. 22, aerosolized lipoplex generated with the vibrating mesh-based nebulizer retained sufficient potency to yield ˜5-fold higher levels of PGE₂ than lipoplex nebulized with the jet nebulizer, with values for the former being slightly lower but similar to values obtained historically for unnebulized lipoplex.

### Example 6

The present example involves integration and validation of airflow sensors into the device of the present disclosure to replace pressure sensors; selection of the respirator synchronizing methodology; and testing of this operational control program for the device of the present disclosure implemented in LabVIEW software.

It is possible to maintain a high level of control over the aerosol delivery parameters in our system by virtue of a computer-based user interface developed for this purpose. This interface makes it possible to observe ventilator patterns and to control the points during respiration that aerosol generation starts and stops. This has been accomplished to date using pressure sensors integrated into the inspiratory and expiratory limbs of the ventilator circuit. In embodiments of the device of the present disclosure the pressure sensors are replaced with flow sensors. Flow sensors are routinely used to monitor patient breathing in a clinical setting and can be used to calculate tidal volume and air velocity (for a given tubing cross section) independent of sensors contained in the ventilator. The flow sensor provides better quality information for timing aerosol generation vis-à-vis the respiratory cycle. In addition, flow sensors that can be calibrated for different ventilator gases are incorporated since gases other than air may be more effective at transporting aerosols to the lungs in some disease states.

Normal variations in respirator timing and sequencing of aerosol delivery require adjustment to the control parameters to tailor optimal aerosol delivery to changing lung mechanics and ventilation patterns. The parameter adjustments are automatic and accurately predict future respiratory cycles. Historical respirator timing information is used as the basis for continuous adjustments to the delivery parameters. A variety of techniques for extracting respirator timing data from the historical respirator data can be considered such as cycle average, Fourier decomposition, wavelet analysis, and neural network modeling.

Incorporation of a Flow Sensor into the Device of the Present Disclosure

During the bronchoconstriction studies described above it appeared that pressure sensors did not respond as accurately under these conditions as under normal airway conditions due to erratic pressure swings in the ventilator circuit that on occasion triggered the aerosol inappropriately. Thus, flow sensors are incorporated in embodiments of the system of the present disclosure to accommodate for such conditions. In the embodiment of the present example one pressure sensor is retained in the ventilator circuit for two purposes: 1) to allow more accurate compensation of air flow measurements using the flow meters discussed below and 2) to provide an independent indicator of patient airway mechanics during ventilation.

A high performance linear OEM mass flow meter (TSI, Incorporated, Shoreview, Minn.) is integrated into the equivalent of a clinical ventilator system. These flow meters utilize a thin platinum film that is heated but cools as the ventilator air passes over it. A small amount of power is applied to the film to maintain its elevated temperature and this power is proportional to flow. The pressure drop across these meters is relatively low (on the order of 6 cm H₂O for the 4120 model at 5 L/min flow, a value typical of flow through adult ventilator circuits). These particular models are available pre-calibrated for air, nitrogen, and oxygen gas flows, and can also accommodate other gases.

Analog output from the meter is input into LabView software to provide a measure of volumetric flow rate and integrated ventilation volume. Additional output measures include pressure and temperature. Further, the software user interface allows a user to select an option for each of the different driving gases to include air, 100% oxygen, mixtures, and other gases.

Development of Computer-Based Program for Prediction of Next Breath

The electronic controller software/computer system component of the present disclosure includes one or more algorithms that incorporate select features of the flow and pressure traces obtained during mechanical ventilation and uses this information to predict optimal times for initiating aerosol generation.

Testing of the Device of the Present Disclosure In Vitro

An in vitro test lung setup such as that shown in FIG. 9 is used to examine the effects of humidity and temperature in a ventilator circuit on the accuracy of the flow sensors and aerosol actuation. As an alternative to the anesthesia machine for ventilation, a T-Bird ventilation system is used and set to deliver air, oxygen, or helium-oxygen mixtures at typical clinical ventilator settings consisting of a tidal volume of 480 ml corresponding to 6 ml/kg for a 80 kg human, a breath rate of 10 breaths/minute, and an inspiratory:expiratory ratio of 1:2. A 0.5 ml dose of ^{99m}Tc-DTPA is delivered using a vibrating mesh-type nebulizer. Aerosol generation is actuated in sync with inspiration and continued for 37.5% of inspiration (corresponding to 1.125 seconds) as these conditions were deemed optimal for aerosol delivery in this system in above examples.

Aerosols are collected in a filter placed between the test lung (“lung” filter) and an endotracheal tube. In addition, aerosol waste is collected in a filter placed in the expiratory limb immediately adjacent to the y-piece (“waste” filter). Flow is then measured with the device of the present disclosure, these measurements are compared to flow measures specified with the ventilator, and variability in the system is determined when using the different gases in temperature-controlled, humidified and non-humidified circuits. Specifically, deposition efficiency is examined after ventilating the test lung with the different gases at 25° C., 31° C., and 37° C. with the humidifier turned on or left off. Deposition efficiency is assessed by counting radioactivity in the “lung” and “waste” filters using a gamma scintillation probe. It is important that the effects of driving gas on deposition is tested to allow more reasonable estimates of aerosol deposition in vivo, particularly during disease states.

### Example 7

The present example involves integration of a waste sensor into the device of the present disclosure to monitor exhaled (wasted) aerosol and provide additional feedback for automatic adjustment of aerosol generation parameters. The electronic controller software includes algorithms that integrate information on breathing pattern and wasted aerosol to determine aerosol delivery patterns that optimize lung deposition and minimize waste.

An optical sensor is incorporated into the device of the present disclosure to allow detection of exhaled aerosol. For optimal aerosol deposition to ventilated patients the tidal volume should be greater than the volume of tubing and the endotracheal tube. To avoid leaving residual aerosol in the tubing it is also important to “chase” the aerosols with a volume substantially equivalent to that of the tubing, and ideally larger to include the large airway dead space in the lung. Due to differences in lung mechanics, optimal aerosol delivery parameters will likely differ among patients and even over time in the same patient. A monitor that senses exhaled aerosol particles provides an on line quantitative indicator of wasted aerosol, permitting a feedback loop through which aerosol production and delivery patterns can be adjusted to optimize the amount of aerosol retained in the lungs.

The amount of isotope not deposited in the lungs and therefore “wasted” in exhaled gas is determined by the aerosol delivery pattern. These exhaled particles will be a mixture of sizes resulting from the size of particles in the delivered aerosol, effects of the humidified, temperature-controlled ventilator circuit, selectivity of deposition by particle size and changes in particle size that occur in vivo. The waste sensor is integrated to monitor these exhaled aerosols. Either of two custom developed aerosol detection techniques can be employed for this purpose: 1) A relatively compact optical system utilizing a CCD camera chip and a pulsed laser custom configured such that the processed output signal provides a measure of the concentrations of aerosols greater than 2 microns in a diameter; 2) A relatively simple and more compact optical “transmissometer” based on a pulsed LED and photodiode that can detect the presence of aerosols in the expiration limb. The amount of radioactive aerosols collected as waste on filters placed in the expiratory limb of the ventilator circuit when using the different driving gases is compared to the amount measured with the particle sensor. The comparison is made using a calibration curve developed for any given nebulizer to associate aerosol particle size with all of the variables that can affect it.

The ability of the system to automatically adjust aerosol delivery parameters, such that exhaled waste is minimized while ventilating at normal airway pressures and after elevations in airway pressure induced by restricting expansion of the test lung, is then tested using a control program that provides a guide for adjusting aerosol generation patterns. The control program includes, but is not limited to, the following steps:
1. The system is started and after 2 minutes of steady ventilation, a baseline waste level is determined over 5 breaths.
2. Aerosol generation is initiated with inspiration and continues for 100% of inspiration, a condition known to cause waste.
3. Based on feedback from the exhalant aerosol monitor and using the flow and pressure data, the nebulization stop time is adjusted.
4. This process is repeated until monitored waste is minimized.

There are a number of techniques available to measure particle size and count them in an air stream. Such techniques have been used in various industrial spray patternization applications and spray combustion characterization. Additionally analysis of variance and unpaired t-tests is used to compare aerosol deposition efficiency under the normal and elevated airway pressure conditions and to compare radioactivity in the “waste” filters to the particle counts.

### Example 8

This present example is designed to demonstrate proof-of-principle related to aerosolized surfactant therapy. Infant piglets (˜2 kg) are randomly assigned to one of five groups including: 1) no intervention group; 2) bolus instillation group; 3) AuContrAer-controlled nebulization group; 4) continuous nebulization group; and 5) saline nebulization group. On the day of the experiment, each piglet is anesthetized, intubated, and connected to an anesthesia machine for subsequent ventilation and maintenance of anesthesia. The right carotid artery is cannulated to allow monitoring of systemic arterial pressure and to allow arterial blood gases to be measured. Room air is used for ventilation at about 30 breaths per minute, with a tidal volume of about 5-10 ml/kg to maintain P_{CO2} at about 35 mm Hg. A small Swan-Ganz catheter is inserted via the right jugular vein into the main pulmonary artery to allow continuous monitoring of pulmonary arterial pressure and core body temperature. The piglet is placed on a heating pad and covered with another heating pad to maintain body temperature at about 39 degrees Celsius.

After allowing the piglet to stabilize for 15 minutes, the average of three static lung compliance measurements is made using a calibrated syringe to inflate the lungs with 5-10 ml/kg of room air while monitoring airway pressure until a pressure plateau is recorded. The area under the curve for pressure is obtained for the time interval of 10-20 seconds after lung inflation. The animal is then ventilated with 100% oxygen for 5 minutes and then lung surfactant depletion is done by repeated lung lavages (30-50 ml) with warmed normal saline. After each lavage the piglet is ventilated with 100% oxygen for 2-5 minutes to minimize hypoxemia. Surfactant depletion is presumed to have occurred when peak airway pressure has more than doubled or when PaO₂ is approximately 80 mm Hg. Ventilation is then switched back to room air for 5 minutes, and blood gases are measured, after which point static lung compliance measurements are repeated.

Radiolabeled surfactant or saline (4 ml/kg) is administered by either bolus instillation, aerosol administration using the system of the present disclosure or continuous nebulization. Blood gas and static lung compliance measurements are repeated 5 minutes after surfactant or saline administration. Instillation procedures are substantially identical to those used in neonatal human infants. Repeat blood gas and compliance measurements are made 1, 2, and 3 hours after surfactant/saline delivery, and then the animals are sacrificed. Delivery efficiency is then assessed using an externally-placed lead-shielded gamma scintillation probe.

We claim:

1. A system for delivery of an aerosolized formulation to the respiratory tract of a host, comprising:
an inspiration sensor operative to perform one or more of the following functions: detect an initiation of respiration, detect a cessation of inspiration, detect a flow rate, and calculate a volume of a gas flowing past the inspiration sensor;
an aerosol generator operative to aerosolize and release a formulation to be delivered to the respiratory tract of the human or animal;
a computer system including an aerosol controller system and in communication with the inspiration sensor and the aerosol generator and operative to perform the following functions:
i. receive information from the inspiration sensor;
ii. process information received from the inspiration sensor and determine at least one respiration parameter;
iii. determine the desired time during a respiration cycle for initiation and cessation of aerosol release based upon one or more respiration parameters and on one or more delivery parameters, wherein the delivery parameters include inputted delivery parameters and calculated delivery parameters, and wherein the computer system determines calculated delivery parameters based on one or more respiration parameters and optionally one or more inputted delivery parameters;
iv. communicate with the aerosol generator to activate and terminate the release of aerosolized formulation;
v. repeat steps i through iv and automatically adjust for any changes in the respiration parameters or delivery parameters; and
vi. repeat i through v until a desired amount of formulation has been delivered.

2. The system of claim 1, wherein the respiration parameters comprise at least one parameter selected from the following:
a length of a respiration cycle, a length of inspiration, a volume of a gas flowing past the inspiration sensor, a peak inspiratory pressure, and a tidal volume.

3. The system of claim 1, wherein the inputted delivery parameters comprise at least one parameter selected from the following:
a patient-specific parameter, a formulation-specific parameter, an airway connection-specific parameter, an estimated dead-space, an estimated chase volume, an estimated gas velocity, an estimated start and stop time for aerosol generation, a specified length of time to complete delivery of a desired volume of formulation, a dosing protocol, and a delivery efficiency setting.

4. The system of claim 1, wherein the calculated delivery parameters comprise one or more of the following:
a calculated dead space, a calculated chase volume, a calculated gas velocity, a calculated particle size, and a calculated start and stop time for aerosol generation.

5. The system of claim 1, wherein the aerosol generator is a metered dose inhaler or a nebulizer.

6. The system of claim 5, wherein the nebulizer is a jet nebulizer or a vibrating mesh nebulizer (should other types be listed?).

7. The system of claim 1, wherein the inspiration sensor is a gas flow sensor.

8. The system of claim 1, further comprising a waste sensor operative to detect an amount of waste, wherein the waste sensor is in communication with the computer system and the computer system receives information from the waste sensor, processes information received from the waste sensor and determines a waste percentage, and automatically adjusts the timing of initiation and cessation of aerosol release if the waste percentage exceeds a pre-determined waste tolerance threshold.

9. The system of claim 8, wherein the pre-determined waste tolerance threshold is between about 0 and 20 percent.

10. The system of claim 8, wherein the waste is selected from one or more of the following types of waste: exhaled waste, wrap-around waste, rain-out waste, and sputter-volume.

11. The system of claim 1, wherein the system is adapted for use with a ventilator.

12. The system of claim 1, wherein the system is adapted for use with a portable metered dose inhaler.

13. The system of claim 1, wherein the aerosolized formulation is a respiratory drug.

14. The system of claim 1, wherein the aerosolized formulation is a drug for treatment of a systemic illness.

15. The system of claim 1, wherein the aerosolized formulation comprises genetic material.

16. The system of claim 1, wherein the aerosolized formulation comprises an imaging composition selected from at least one of: radioisotopes, contrast agents, and labeled pharmaceutical compositions.

17. The system of claim 1, wherein the aerosolized formulation comprises a pulmonary surfactant.

18. The system of claim 1, wherein the computer system automatically records and logs the respiration parameters for a number of consecutive respiration cycles, calculates average respiration parameters from the recorded respiration parameters, and determines the desired time for initiation and cessation of aerosol release based at least in part on the calculated average respiration parameters.

19. The system of claim 18, wherein the computer system calculates average respiration parameters from the recorded respiration parameters of a certain number of respiration cycles having respiration parameters within a determined percentage of one another.

20. The system of claim 18, wherein the computer system continuously re-calculates the average respiration parameters based on new information received from the inspiration sensor to obtain adjusted average respiration parameters, and uses the adjusted average respiration parameters to adjust calculated delivery parameters and to adjust the desired time for initiation and cessation of aerosol release to accommodate for the adjusted average respiration parameters.

21. The system of claim 3, wherein the patient-specific parameters are selected from one or more of the following:
a height, a weight, an age, a gender, a desired location in respiratory tract for delivery of the aerosolized particles, a respiratory condition, and an estimated tidal volume.

22. The system of claim 3, wherein the formulation-specific parameters are selected from one or more of the following:
a volume of a formulation to be delivered, a type of formulation to be delivered, a pre-determined size of particles of the aerosolized formulation, a pre-determined gas velocity of the aerosolized formulation.

23. The system of claim 3, wherein the airway connection-specific parameters are selected from one or more of the following:
an airway connection tube diameter, and an airway connection tube volume.

24. The system of claim 1, further comprising an efficiency controller operative to select a desired efficiency setting.

25. The system of claim 1, wherein the system is able to monitor and deliver more than one different type of formulation, wherein the calculated delivery parameters for each formulation may be different.

26. A system for delivery of an aerosolized formulation to the respiratory tract of a ventilated host, comprising:
a ventilator, to which the ventilated host is attached;
an inspiration sensor in communication with an inspiratory pathway of the ventilator and operative to perform one or more of the following functions: detect an initiation of respiration, detect a cessation of inspiration, detect a flow rate, and calculate a volume of a gas flowing past the inspiration sensor;
an aerosol generator operative to aerosolize and release a formulation to be delivered to the respiratory tract of the host, wherein said aerosol generator is in communication with the inspiratory pathway of the ventilator to allow release of the aerosolized formulation from the aerosol generator into the inspiratory pathway;
a waste sensor in communication with an expiratory pathway of the ventilator operative to detect an amount of exhaled waste;
a computer system in communication with the inspiration sensor, waste sensor, and the aerosol generator operative to perform the following functions:
i. receive information from the inspiration sensor and the waste sensor;
ii. process information received from the inspiration sensor and determine at least one respiration parameter;
iii. process information received from the waste sensor and determine a waste percentage;
iv. determine a desired time during a respiration cycle for initiation and cessation of aerosol release based upon one or more respiration parameters and on one or more delivery parameters, wherein the delivery parameters include inputted delivery parameters and calculated delivery parameters, and wherein the computer system determines calculated delivery parameters based on one or more respiration parameters and optionally one or more inputted delivery parameters and optionally the waste percentage;
v. communicate with the aerosol generator to activate and terminate the release of aerosolized formulation;
vii. repeat steps i through v and automatically adjust for any changes in the respiration parameters or delivery parameters; and
viii. repeat steps i through vii until a desired amount of formulation has been delivered.

27. A method for optimizing delivery of an aerosolized formulation to the respiratory tract of a human or animal, comprising:
a. detecting respiration data;
b. determining respiration parameters from respiration data;
c. determining calculated delivery parameters from respiration data and one or more inputted delivery parameters;
d. determining the desired time during a respiration cycle for initiation and cessation of aerosol release based upon one or more respiration parameters and on one or more calculated delivery parameters;
e. communicating with an aerosol generator to activate and terminate the release of aerosolized formulation;
f. repeating steps a through e and adjusting for any changes in the respiration parameters or delivery parameters; and
g. repeating a through f until a desired amount of formulation has been delivered.

28. The method of claim 27, wherein the respiration data includes at least one of the following: a time of initiation of inspiration, a time of cessation of inspiration, a rate of a gas being inhaled, and a volume of a gas being inhaled.

29. The method of claim 27, wherein inputted delivery parameters are selected from one or more of the following parameters: a patient-specific parameter, a formulation-specific parameter, an airway connection-specific parameter, a dosing protocol, a specified length of time to complete delivery of a desired volume of formulation, and a delivery efficiency setting

30. The method of claim 27, wherein the respiration parameters comprise at least one parameter selected from the following:
A length of a respiration cycle, a length of inspiration, a peak inspiratory pressure, and a tidal volume.

31. The method of claim 27, wherein the calculated delivery parameters comprise one or more of the following:
a calculated dead space, a calculated chase volume, a calculated gas velocity, a calculated particle size, and a calculated start and stop time for aerosol generation.

32. The method of claim 27, further comprising the following steps:
a. detecting an amount of waste,
b. determining a waste percentage, and
c. re-calculating one or more calculated delivery parameters
if the waste percentage exceeds a pre-determined waste tolerance threshold.

33. The method of claim 32, wherein the pre-determined waste tolerance threshold is between about 0 and 20 percent.

34. The method of claim 32, wherein the waste is selected from one or more of the following types of waste: exhaled waste, wrap-around waste, rain-out waste, and sputter-volume.

35. The method of claim 27, wherein the patient-specific parameters are selected from one or more of the following:
a height, a weight, an age, a gender, a desired location in respiratory tract for delivery of the aerosolized particles, a respiratory condition, and an estimated tidal volume.

36. The method of claim 27, wherein the formulation-specific parameters are selected from one or more of the following:
a volume of a formulation to be delivered, a type of formulation to be delivered, a pre-determined size of particles of the aerosolized formulation, a pre-determined gas velocity of the aerosolized formulation.

37. The method of claim 27, wherein the airway connection-specific parameters are selected from one or more of the following:
an airway connection tube diameter, and an airway connection tube volume.

38. The method of claim 27, wherein the determining respiration parameters from respiration data includes recording and logging the respiration parameters for a number of consecutive respiration cycles, calculating average respiration parameters from the recorded respiration parameters, and wherein determining the desired time for initiation and cessation of aerosol release is based at least in part on the calculated average respiration parameters.

39. The method of claim 37, wherein the average respiration parameters are calculated from the recorded respiration parameters of a certain number of respiration cycles having respiration parameters within a determined percentage of one another.

40. The method of claim 37, further comprising continuously re-calculating the average respiration parameters based on continuously acquired respiration data to obtain adjusted average respiration parameters, and using the adjusted average respiration parameters to adjust calculated delivery parameters and to adjust the desired time for initiation and cessation of aerosol release to accommodate for the adjusted average respiration parameters.

41. A method for optimizing delivery of an aerosolized formulation to the respiratory tract of a human or animal, comprising:
a. acquiring inputted delivery parameters, wherein inputted delivery parameters are selected from one or more of the following parameters: a patient-specific parameter, a formulation-specific parameter, an airway connection-specific parameter, an estimated start and stop time for aerosol generation, a specified length of time to complete delivery of a desired volume of formulation, and a delivery efficiency setting;
b. determining calculated delivery parameters from one or more inputted delivery parameters;
c. determining the desired time during a respiration cycle for initiation and cessation of aerosol release based upon one or more calculated delivery parameters;
d. communicating with an aerosol generator to activate and terminate the release of aerosolized formulation;
e. detecting an amount of waste selected from wrap-around waste and exhaled waste;
f. adjusting calculated delivery parameters with respect to the amount of waste detected;
g. repeating steps a through f and automatically adjusting for any changes in the amount of waste detected or the delivery parameters; and
h. repeating a through g until a desired amount of formulation has been delivered.

42. A system comprising:
an aerosol controller logic configured to:
determine a desired time during a respiration cycle for initiation and cessation of aerosol generation based upon one or more calculated delivery parameters calculated from one or more respiration parameters and one or more inputted delivery parameters, and optionally a waste percentage; and
automatically adjust the desired time for initiation and cessation of aerosol generation based upon any changes in calculated delivery parameters.

43. The system of claim 41 further comprising a processor configured to execute the aerosol controller logic of claim 41.

44. The system of claim 41, wherein the aerosol controller logic is stored on a computer readable medium.

45. The system of claim 41, wherein the aerosol controller logic comprises software, hardware, or a combination of software and hardware.
